(19) United States
(12) Patent Application Publication
(10) Pub. No.: US 2007/0015757 A1
(43) Pub. Date: Jan. 18, 2007

(54) Novel Glucagon Antagonists/Inverse Agonists

(75) Inventors: Peter Madsen, Bagsvaerd (DK); Jesper Lau, Farum (DK); Janos Tibor Kodra, Kobenhavn O (DK); Inge Thoger Christensen, Kgs. Lyngby (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/424,871

(22) Filed: Jun. 18, 2006

Related U.S. Application Data
(63) Continuation of application No. PCT/EP04/53580, filed on Dec. 17, 2004.
(60) Provisional application No. 60/531,733, filed on Dec. 22, 2003.

(30) Foreign Application Priority Data
Dec. 19, 2003 (DK) PA 2003 01894

(51) Int. Cl. A61K 31/53; A61K 31/50; A61K 31/505; A61K 31/4965; A61K 31/455; A61K 31/433; A61K 31/426; A61K 31/425; A61K 31/421; A61K 31/42; A61K 31/4196; A61K 31/4192; A61K 31/415; A61K 31/381; A61K 31/35; A61K 31/34; A61K 31/198

(52) U.S. Cl. 514/241; 514/381; 514/247; 514/252.1; 514/256; 514/563; 514/383; 514/362; 514/364; 514/365; 514/374; 514/355; 514/372; 514/379; 514/396; 514/438; 514/459; 514/242; 544/183; 544/208; 544/330; 544/406; 546/315; 548/200; 548/123; 548/206; 548/236; 548/248; 548/253; 548/266.8; 549/76; 562/450

(57) ABSTRACT

Novel compounds that act to antagonize the action of the glucagon peptide hormone on the glucagon receptor. More particularly, it relates to glucagon antagonists or inverse agonists.

### CROSS-REFERENCE TO RELATED PATENT APPLICATIONS

This patent application is a continuation of International Patent Application PCT/EP2004/053580, filed Dec. 17, 2004 (published as WO 2005/058845), which designates the US, and claims the benefit of U.S. Provisional Patent Application 60/531,733, filed Dec. 22, 2003, and Danish Patent Application PA 2003 01894, filed Dec. 19, 2003, the entirety of each of which is hereby incorporated by reference.

### FIELD OF THE INVENTION

The present invention relates to agents that act to antagonize the action of the glucagon peptide hormone on the glucagon receptor. More particularly, it relates to glucagon antagonists or inverse agonists.

### BACKGROUND OF THE INVENTION

Glucagon is a key hormonal agent that, in co-operation with insulin, mediates homeostatic regulation of the amount of glucose in the blood. Glucagon primarily acts by stimulating certain cells (mostly liver cells) to release glucose when blood glucose levels fall. The action of glucagon is opposite to that of insulin, which stimulates cells to take up and store glucose whenever blood glucose levels rise. Both glucagon and insulin are peptide hormones. Glucagon is produced in the alpha islet cells of the pancreas and insulin in the beta islet cells. Diabetes mellitus is a common disorder of glucose metabolism. The disease is characterized by hyperglycemia and may be classified as type 1 diabetes, the insulin-dependent form, or type 2 diabetes, which is non-insulin-dependent in character. Subjects with type 1 diabetes are hyperglycemic and hypoinsulinemic, and the conventional treatment for this form of the disease is to provide insulin. However, in some patients with type 1 or type 2 diabetes, absolute or relative elevated glucagon levels have been shown to contribute to the hyperglycemic state. Both in healthy control animals as well as in animal models of type 1 and type 2 diabetes, removal of circulating glucagon with selective and specific antibodies has resulted in reduction of the glycemic level. These studies suggest that glucagon suppression or an action that antagonizes glucagon could be a useful adjunct to conventional treatment of hyperglycemia in diabetic patients. The action of glucagon can be suppressed by providing an antagonist or an inverse agonist, ie substances that inhibit or prevent glucagon-induced responses. The antagonist can be peptidic or non-peptidic in nature.

Native glucagon is a 29 amino acid peptide having the sequence:

His-Ser-Glu-Gly-Thr-Phe-Thr-Ser-Asp-Tyr-Ser-Lys-Tyr-Leu-Asp-Ser-Arg-Arg-Ala-Gln-AspPhe-Val-Gln-Trp-Leu-Met-Asn-Thr-OH

Glucagon exerts its action by binding to and activating its receptor, which is part of the Glucagon-Secretin branch of the 7-transmembrane G-protein coupled receptor family. The receptor functions by activating the adenylyl cyclase second messenger system and the result is an increase in cAMP levels.

Several publications disclose peptides that are stated to act as glucagon antagonists. Probably, the most thoroughly characterized antagonist is DesHis¹[Glu⁹]-glucagon amide (Unson et al., Peptides 10, 1171 (1989); Post et al., Proc. Natl. Acad. Sci. USA 90, 1662 (1993)). Other antagonists are DesHis¹,Phe⁶[Glu⁹]-glucagon amide (Azizh et al., Bioorganic & Medicinal Chem. Lett. 16, 1849 (1995)) and NLeu⁹,Ala^{11,16}-glucagon amide (Unson et al., J. Biol. Chem. 269 (17), 12548 (1994)).

Peptide antagonists of peptide hormones are often quite potent. However, they are generally known not to be orally available because of degradation by physiological enzymes, and poor distribution in vivo. Therefore, orally available non-peptide antagonists of peptide hormones are generally preferred. Among the non-peptide glucagon antagonists, a quinoxaline derivative, (2-styryl-3-[3-(dimethylamino)propylmethylamino]-6,7-dichloroquinoxaline was found to displace glucagon from the rat liver receptor (Collins, J. L. et al., Bioorganic and Medicinal Chemistry Letters 2(9):915-918 (1992)). WO 94/14426 (The Wellcome Foundation Limited) discloses use of skyrin, a natural product comprising a pair of linked 9,10-anthracenedione groups, and its synthetic analogues, as glucagon antagonists. U.S. Pat. No. 4,359,474 (Sandoz) discloses the glucagon inhibiting properties of 1-phenyl pyrazole derivatives. U.S. Pat. No. 4,374,130 (Sandoz) discloses substituted disilacyclohexanes as glucagon inhibiting agents. WO 98/04528 (Bayer Corporation) discloses substituted pyridines and biphenyls as glucagon antagonists. U.S. Pat. No. 5,776,954 (Merck & Co., Inc.) discloses substituted pyridyl pyrroles as glucagon antagonists and WO 98/21957, WO 98/22108, WO 98/22109 and U.S. Pat. No. 5,880,139 (Merck & Co., Inc.) disclose 2,4-diaryl-5-pyridylimidazoles as glucagon antagonists. Furthermore, WO 97/16442 and U.S. Pat. No. 5,837,719 (Merck & Co., Inc.) disclose 2,5-substituted aryl pyrroles as glucagon antagonists. WO 98/24780, WO 98/24782, WO 99/24404 and WO 99/32448 (Amgen Inc.) disclose substituted pyrimidinone and pyridone compounds and substituted pyrimidine compounds, respectively, which are stated to possess glucagon antagonistic activity. Madsen et al. (J. Med. Chem. 41, 5151-7 (1998)) discloses a series of 2-(benzimidazol-2-ylthio)-1-(3,4-dihydroxyphenyl)-1-ethanones as competitive human glucagon receptor antagonists. WO 99/01423 and WO 00/39088 (Novo Nordisk A/S) disclose different series of alkylidene hydrazides as glucagon antagonists/inverse agonists. WO 00/69810, WO 02/00612, WO 02/40444, WO 02/40445 and WO 02/40446 (Novo Nordisk A/S) disclose further classes of glucagon antagonists.

These known glucagon antagonists differ structurally from the present compounds.

### SUMMARY OF THE INVENTION

The invention provides compounds of the general formula (I): 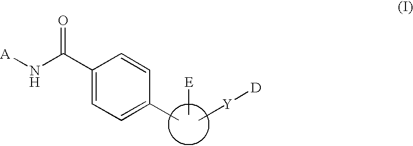
wherein
A is 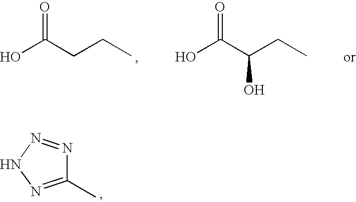
Y is a valence bond, >C═O, ═CR¹—, —(CR¹R²)ₘ—, —NR¹—, ═N—,
wherein R¹ and R² are independently selected from H and lower alkyl;
m is selected from 1, 2, 3, 4, 5 or 6;
E is
C₁₋₁₀-alkyl or C₂₋₁₀-alkenyl,
C₃₋₁₀-cycloalkyl, C₃₋₁₀-cycloalkenyl, C₇₋₁₀-bicycloalkyl, C₃₋₁₀-cycloalkyl-C₁₋₆-alkyl, C₃₋₁₀-cycloalkenyl-C₁₋₆-alkyl or C₇₋₁₀-bicycloalkyl-C₁₋₆-alkyl,
wherein the rings may optionally be substituted with one or more substituents selected from halogen, C₁₋₆-alkyl, C₂₋₆-alkenyl, C₁₋₆-alkoxy, C₁₋₆-thioalkyl, —CF₃, —OCF₃, —SCF₃, —OCHF₂ and —SCHF₂,
aryl, aryloxy, arylthio, heteroaryl, aryl-C₁₋₆-alkyl, aryloxy-C₁₋₆-alkyl, arylthio-C₁₋₆-alkyl, heteroaryl-C₁₋₆-alkyl, diaryl-C₁₋₆-alkyl or (C₁₋₆-alkyl)(aryl)-C₁₋₇-alkyl, wherein the non-aromatic and aromatic rings may optionally be substituted with one or more substituents selected from halogen, C₁₋₆-alkyl, C₂₋₆-alkenyl, C₁₋₆-alkoxy, C₁₋₆-thioalkyl, —CF₃, —OCF₃, —SCF₃, —OCHF₂, —SCHF₂, C₃₋₁₀-cycloalkyl and C₃₋₁₀-cyclo-alkenyl, or with two substituents on adjacent positions which are combined to form a bridge C₁₋₆-alkylene, C₂₋₆-alkenylene or —O—C₁₋₆-alkylene-O—, 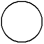
represents a phenyl, C₃₋₈-cycloalkyl, or a 4-, 5-, 6- or 7-membered heterocycle,
   D is aryl or heteroaryl,
   which may optionally be substituted with one or more substituents selected from
halogen, —CF₃, —OCF₃, —SCF₃, —CN, —NO₂, C₁₋₁₀-alkyl, C₂₋₆-alkenyl, C₁₋₆-alkoxy, C₁₋₆-alkylthio, amino, C₁₋₆-alkylamino, di-C₁₋₆-alkylamino, —SO₂CF₃ and —SO₂—C₁₋₆-alkyl,
C₃₋₈-cycloalkyl, C₃₋₈-cycloalkenyl, aryl and aryl-C₁₋₆-alkoxy, wherein the non-aromatic and aromatic rings optionally may be substituted with one to three substituents selected from halogen, —CF₃, —OCF₃, —SCF₃, —CN, —NO₂, C₁₋₁₀-alkyl, C₂₋₆-alkenyl, C₁₋₆-alkoxy and C₁₋₆-alkylthio, or with two substituents on adjacent positions which are combined to form a bridge —O—(CH₂)₈—O—(CH₂)ₚ— or —O—(CF₂)ₙ—O—(CF₂)ₚ—, wherein s is an integer of from 1 to 6, and p is 0 or 1,
or with two substituents on adjacent positions which are combined to form a bridge —O—(CH₂)ₛ—O—(CH₂)ₚ— or —O—(CF₂)ₛ—O—(CF₂)ₚ—, wherein s is an integer of from 1 to 6, and p is 0 or 1,
   as well as any diastereomer or enantiomer or tautomeric form thereof including mixtures of these or a pharmaceutically acceptable salt thereof.

In a particular aspect of the invention, the invention related to compounds according to formula (I) as above wherein

E is

C₁₋₁₀-alkyl or C₂₋₁₀-alkenyl,
C₃₋₁₀-cycloalkyl or C₃₋₁₀-cycloalkenyl, which may optionally be substituted with one or two substituents selected from halogen, C₁₋₆-alkyl, C₁₋₆-alkoxy, C₁₋₆-thioalkyl, —CF₃, —OCF₃, —SCF₃, —OCHF₂ and —SCHF₂, 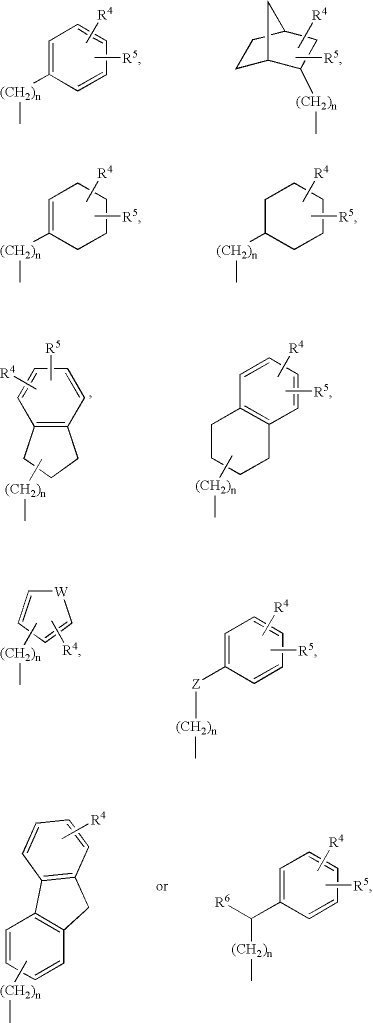
R⁴ and R⁵ independently are hydrogen, halogen, C₁₋₆-alkyl, C₂₋₆-alkenyl, C₁₋₆-alkoxy, C₁₋₆-thioalkyl, —CF₃, —OCF₃, —SCF₃, —OCHF₂, —SCHF₂, C₃₋₁₀-cycloalkyl or C₃₋₁₀-cyclo-alkenyl, or R⁴ and R⁵ on adjacent positions may be combined to form a bridge —O—C₁₋₆-alkylene-O—, C₁₋₈-alkylene or C₃₋₈-alkenylene,
R⁶ is C₁₋₆-alkyl or aryl, wherein aryl may optionally be substituted with one or two substituents selected from halogen, C₁₋₆-alkyl, C₂₋₆-alkenyl, C₁₋₆-alkoxy, C₁₋₆-thioalkyl, —CF₃, —OCF₃, —SCF₃, —OCHF₂ and —SCHF₂,
n is an integer of from 0 to 6,
Z is —O— or —S—,
W is —O—, —S—, or —NR⁷—,
R⁷ is hydrogen or C₁₋₆-alkyl,
   D is 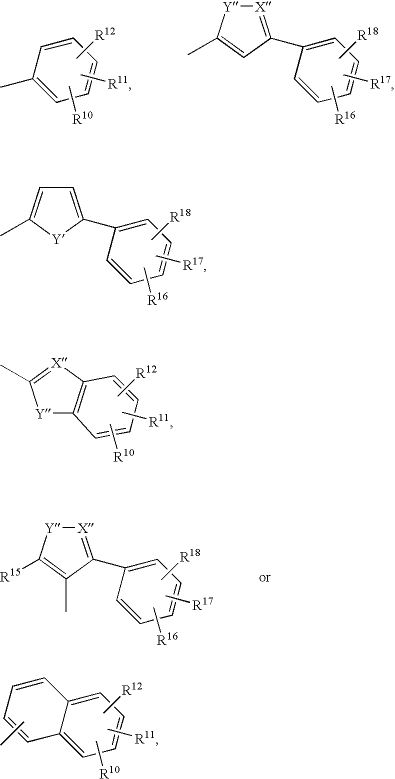
   R¹⁰, R¹¹ and R¹² independently are
hydrogen, halogen, —CF₃, —OCF₃, —SCF₃, —CN, —NO₂, C₁₋₁₀-alkyl, C₂₋₆-alkenyl, C₁₋₆-alkoxy, C₁₋₆-alkylthio, amino, C₁₋₆-alkylamino, di-C₁R₆-alkylamino, —SO₂CF₃ or —SO₂—C₁₋₆-alkyl,
C₃₋₈-cycloalkyl, C₃₋₈-cycloalkenyl, aryl or aryl-C₁₋₆-alkoxy,
wherein the non-aromatic and aromatic rings optionally may be substituted with one to three substituents selected from halogen, —CF₃, —OCF₃, —SCF₃, —CN, —NO₂, C₁₋₁₀-alkyl, C₂₋₆-alkenyl, C₁₋₆-alkoxy and C₁₋₆-alkylthio, or with two substituents on adjacent positions which are combined to form a bridge —O—(CH₂)₈—O—(CH₂)ₚ— or —O—(CF₂)ₙ—O—(CF₂)ₚ—, wherein s is an integer of from 1 to 6, and p is 0 or 1,
or two of R¹⁰, R¹¹ and R¹² on adjacent positions are combined to form a bridge —O—(CH₂)ₛ—O—(CH₂)ₚ— or —O—(CF₂)ₛ—O—(CF₂)ₚ—, wherein s is an integer of from 1 to 6, and p is 0 or 1,
   X″ is —N═or —CR¹³═
   Y″ is —S—, —O— or —NR¹⁴—,
   —R¹³ and R¹⁵ independently are hydrogen, C₁₋₆-alkyl or aryl, wherein aryl is optionally substituted with one or two substituents selected from halogen, C₁₋₆-alkyl, C₁₋₆-alkoxy, C₁₋₆-thioalkyl, —CF₃, —OCF₃, —SCF₃, —OCHF₂ and —SCHF₂,
   R¹⁴ is hydrogen or C₁₋₆-alkyl,
   R¹⁶, R¹⁷ and R¹ independently are hydrogen, halogen, —CF₃, —OCF₃, —SCF₃, —CN, —NO₂, C₁₋₁₀-alkyl, C₂₋₆-alkenyl, C₁₋₆-alkoxy and C₁₋₆-alkylthio, or with two substituents on adjacent positions which are combined to form a bridge —O—(CH₂)_{q}—O—(CH₂)ᵣ— or —O(CF₂)_{q}—O—(CF₂)ᵣ—, wherein q is an integer of from 1 to 6, and r is 0 or 1,
   as well as any diastereomer or enantiomer or tautomeric form thereof including mixtures of these or a pharmaceutically acceptable salt thereof.

Another aspect of the invention provides compounds of the general formula (I₁): 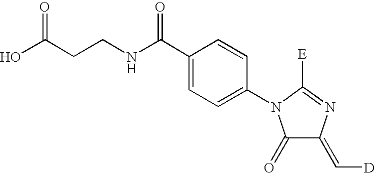
wherein E and D are as defined above.

Another aspect of the invention provides compounds of the general formula (I₃): 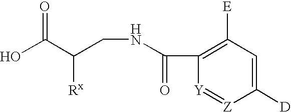
wherein R^{x} represents H or OH, and —Y=Z- (or ═Y-Z=) is —N═N- (or ═N—N═), —O—, —S—, —NR′—, wherein R′ is hydrogen, lower alkyl, lower alkoxy, hydroxy, amino, lower alkylaryl, or aryl and E and D are as defined above, as well as any diastereomer or enantiomer or tautomeric form thereof including mixtures of these or a pharmaceutically acceptable salt thereof.

Another aspect of the invention provides compounds of the general formula (I₄): 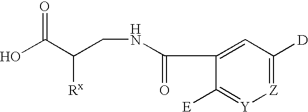
wherein Rx represents H or OH, and —Y=Z- (or ═Y-Z=) is —N═N- (or ═N—N═), —O—, —S—, —NR′—, wherein R′ is hydrogen, lower alkyl, lower alkoxy, hydroxy, amino, lower alkylaryl, or aryl and E and D are as defined above, as well as any diastereomer or enantiomer or tautomeric form thereof including mixtures of these or a pharmaceutically acceptable salt thereof.

Another aspect of the invention provides compounds of the general formula: 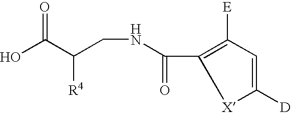
wherein X′ is —O—, —S—, —NR′—, wherein R′ is hydrogen, lower alkyl, lower alkoxy, hydroxy, amino, lower alkylaryl, or aryl and E and D are as defined above, as well as any diastereomer or enantiomer or tautomeric form thereof including mixtures of these or a pharmaceutically acceptable salt thereof.

An aspect of the invention provides compounds as above, which has an IC₅₀ value of no greater than 5 μM as determined by the Glucagon Binding Assay (I) or Glucagon Binding Assay (II) disclosed herein.

An aspect of the invention provides compounds as above, which is an agent useful for the treatment of an indication selected from the group consisting of hyperglycemia, IGT, type 2 diabetes, type 1 diabetes, dyslipidemia and obesity.

An aspect of the invention provides compounds as above for use as a medicament.

The invention provides pharmaceutical compositions comprising, as an active ingredient, at least one compound as above together with one or more pharmaceutically acceptable carriers or excipients.

The invention also provides use of a compound as above for the preparation of a medicament for the treatment of disorders or diseases, wherein a glucagon antagonistic action is beneficial.

The invention provides a method for the treatment of disorders or diseases, wherein a glucagon antagonistic action is beneficial, the method comprising administering to a subject in need thereof an effective amount of a compound as above or a pharmaceutical composition as mentioned above.

Definitions

The following is a detailed definition of the terms used to describe the compounds of the invention:

“Halogen” designates an atom selected from the group consisting of F, Cl, Br and I.

The term “C₁₋₆-alkyl” as used herein represents a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

In the same way “C₁₋₁₀-alkyl” denotes a saturated, branched or straight hydrocarbon group having from 1 to 10 carbon atoms.

The term “C₂₋₆-alkenyl” as used herein represents a branched or straight hydrocarbon group having from 2 to 6 carbon atoms and at least one double bond. Examples of such groups include, but are not limited to, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

In the same way “C₂₋₁₀-alkenyl” denotes a saturated, branched or straight hydrocarbon group having from 2 to 10 carbon atoms.

The term “C₁₋₆-alkoxy” as used herein refers to the radical —O—C₁₋₆-alkyl, wherein C₁₋₆-alkyl is as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “C₁₋₆-alkylthio” as used herein refers to the radical —S—C₁₋₆-alkyl, wherein C₁₋₆-alkyl is as defined above. Representative examples include, but are not limited to, methylthio, ethylthio, n-propylthio, isopropylthio, butylthio, isobutylthio, sec-butylthio, tert-butylthio, n-pentylthio, isopentylthio, neopentylthio, tert-pentylthio, n-hexylthio, isohexylthio and the like.

The term “C₃₋₁₀-cycloalkyl” as used herein represents a saturated, carbocyclic group having from 3 to 10 carbon atoms. Representative examples are cyclopropyl, cyclobutyl, cyclopentyl, cyclohexyl, cycloheptyl, cyclooctyl, cyclononyl, cyclodecyl and the like.

The term “C₇₋₁₀-bicycloalkyl” as used herein represents a bicyclic, saturated, carbocyclic group having from 7 to 10 carbon atoms. Representative examples are bicyclo[3.1.0]hexyl, bicyclo[4.1.0]heptyl, bicyclo[3.1.1]heptyl, bicyclo[2.2.1]heptyl and the like.

The term “C₃₋₁₀-cycloalkenyl” as used herein represents a non-aromatic, carbocyclic group having from 3 to 10 carbon atoms containing one or two double bonds. Representative examples are 1-cyclopentenyl, 2-cyclopentenyl, 3-cyclopentenyl, 1-cyclohexenyl, 2-cyclohexenyl, 3-cyclohexenyl, 2-cycloheptenyl, 3-cycloheptenyl, 2-cyclooctenyl, 1,4-cyclooctadienyl, 1-cyclononenyl, 2-cyclononenyl, 1-cyclodocenyl, 2-cyclodocenyl, and the like.

The term “aryl” as used herein is intended to include carbocyclic, aromatic ring systems such as 6 membered monocyclic and 9 to 14 membered bi- and tricyclic, carbocyclic, aromatic ring systems. Representative examples are phenyl, biphenylyl, naphthyl, anthracenyl, phenanthrenyl, fluorenyl, indenyl, azulenyl and the like. Aryl is also intended to include the partially hydrogenated derivatives of the ring systems enumerated above. Non-limiting examples of such partially hydrogenated derivatives are 1,2,3,4-tetrahydronaphthyl, 1,4-dihydronaphthyl, indanyl and the like.

The term “aryloxy” as used herein denotes a group —O-aryl, wherein aryl is as defined above.

The term “arylthio” as used herein denotes a group —S-aryl, wherein aryl is as defined above.

“Aryl-C₁₋₆-alkyl”, “heteroaryl-C₁₋₆-alkyl”, “aryl-C₂₋₆-alkenyl” etc. mean C₁₋₆-alkyl or C₂₋₆-alkenyl as defined above, substituted by an aryl or heteroaryl as defined above, for example:

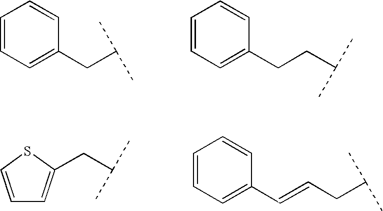

The term “, -5, 6- and 7-membered heterocycle” as used herein is intended to include aromatic as well as fully or partially saturated monocyclic heterocyclic ring systems containing one or more heteroatoms selected from nitrogen, oxygen and sulfur and the rings are optionally substituted with one or two substituents selected from C₁₋₆-alkyl or hydroxy (which may give a keto-group depending on tautomerisme). Representative examples are furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl and thiadiazinyl, including the fully or partially saturated analogues and alkyl- and hydroxy substituted derivatives of any of the above.

The term “heteroaryl” as used herein is intended to include aromatic, heterocyclic ring systems containing one or more heteroatoms selected from nitrogen, oxygen and sulfur such as 5 to 7 membered monocyclic and 8 to 14 membered bi- and tricyclic aromatic, heterocyclic ring systems containing one or more heteroatoms selected from nitrogen, oxygen and sulfur. Representative examples are furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the ring systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The term “optionally substituted” as used herein means that the groups in question are either unsubstituted or substituted with one or more of the substituents specified. When the groups in question are substituted with more than one substituent the substituents may be the same or different.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

### DESCRIPTION OF THE INVENTION

The compounds of the present invention may be chiral, and it is intended that any enantiomers, as separated, pure or partially purified enantiomers or racemic mixtures thereof are included within the scope of the invention.

Furthermore, when a double bond or a fully or partially saturated ring system or more than one center of asymmetry or a bond with restricted rotatability is present in the molecule diastereomers may be formed. It is intended that any diastereomers, as separated, pure or partially purified diastereomers or mixtures thereof are included within the scope of the invention.

Furthermore, some of the compounds of the present invention may exist in different tautomeric forms and it is intended that any tautomeric forms, which the compounds are able to form, are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methyl-, dimethyl-, trimethyl-, ethyl-, hydroxyethyl-, diethyl-, butyl-, tetramethylammonium salts and the like.

Also intended as pharmaceutically acceptable acid addition salts are the hydrates, which the present compounds, are able to form.

Furthermore, the pharmaceutically acceptable salts comprise basic amino acid salts such as lysine, arginine and ornithine.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming pharmacologically active substances. In general, such prodrugs will be functional derivatives of present compounds, which are readily convertible in vivo into the required compound. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The compounds according to the present invention act to antagonize the action of glucagon and are accordingly useful for the treatment of disorders and diseases in which such an antagonism is beneficial.

The compounds according to the invention preferably have an IC₅₀ value of no greater than 5 μM as determined by the Glucagon Binding Assay (I) or Glucagon Binding Assay (II) disclosed herein.

More preferably, the compounds according to the invention have an IC₅₀ value of less than 1 μM, preferably of less than 500 nM and even more preferred of less than 100 nM as determined by the Glucagon Binding Assay (I) or Glucagon Binding Assay (II) disclosed herein.

Furthermore, the compounds according to the invention preferably have a higher binding affinity to the glucagon receptor than to the GIP receptor.

Accordingly, the present compounds may be applicable for the treatment of hyperglycemia, hyper insulinemia, beta-cell rest, improved beta-cell function by restoring first phase response, prandial hyperglycemia, preventing apoptosis, IFG, metabolic syndrome, hypoglycemia, hyper-/hypokalemia, normalising glucagon levels, improved LDL/HDL ratio, reducing snacking, eating disorders, weight loss, PCOS, obesity as a consequence of diabetes, LADA, insulitis, islet transplantation, pediatric diabetes, gestational diabetes, diabetic late complications, micro-/macroalbuminuria, nephropathy, retinopathy, neuropathy, diabetic foot ulcers, reduced intestinal motility due to glucagon administration, short bowel syndrome, antidiarrheic, increasing gastric secretion, decreased blood flow, erectile dysfunction (male & female), glaucoma, post surgical stress, ameliorating organ tissue injury caused by reperfusion of blood flow after ischaemia, ischemic heart damage, heart insufficiency, congestional heart failure, stroke, myocardial infarction, arrythmia, premature death, anti-apoptosis, wound healing, IGT (impaired glucose tolerance), insulin resistance syndromes, syndrome X, type 1 diabetes, type 2 diabetes, hyperlipidemia, dyslipidemia, hypertriglyceridemia, hyperlipoproteinemia, hypercholesterolemia, arteriosclerosis including atherosclerosis, glucagonomas, acute pancreatitis, cardiovascular diseases, hypertension, cardiac hypertrophy, gastrointestinal disorders, obesity, diabetes as a consequence of obesity, diabetic dyslipidemia, etc.

Furthermore, they may be applicable as diagnostic agents for identifying patients having a defect in the glucagon receptor, as a therapy to increase gastric acid secretions and to reverse intestinal hypomobility due to glucagon administration.

They may also be useful as tool or reference molecules in labelled form in binding assays to identify new glucagon antagonists.

Accordingly, in a further aspect the invention relates to a compound according to the invention for use as a medicament.

The invention also relates to pharmaceutical compositions comprising, as an active ingredient, at least one compound according to the invention together with one or more pharmaceutically acceptable carriers or excipients.

The pharmaceutical composition is preferably in unit dosage form, comprising from about 0.05 mg to about 1000 mg, preferably from about 0.1 mg to about 500 mg and especially preferred from about 0.5 mg to about 200 mg of the compound according to the invention.

Furthermore, the invention relates to the use of a compound of the general formula (I) as well as any diastereomer or enantiomer or tautomeric form thereof including mixtures of these or a pharmaceutically acceptable salt thereof for the preparation of a medicament for the treatment of disorders or diseases, wherein a glucagon antagonistic action is beneficial.

The invention also relates to a method for the treatment of disorders or diseases, wherein a glucagon antagonistic action is beneficial the method comprising administering to a subject in need thereof an effective amount of a compound according to the invention.

In one embodiment of the invention the present compounds are used for the preparation of a medicament for the treatment of any glucagon-mediated conditions and diseases.

In another embodiment of the invention the present compounds are used for the preparation of a medicament for the treatment of hyperglycemia.

In yet another embodiment of the invention the present compounds are used for the preparation of a medicament for lowering blood glucose in a mammal. The present compounds are effective in lowering the blood glucose, both in the fasting and the postprandial stage.

In still another embodiment of the invention the present compounds are used for the preparation of a pharmaceutical composition for the treatment of IGT.

In a further embodiment of the invention the present compounds are used for the preparation of a pharmaceutical composition for the treatment of type 2 diabetes.

In yet a further embodiment of the invention the present compounds are used for the preparation of a pharmaceutical composition for the delaying or prevention of the progression from IGT to type 2 diabetes.

In yet another embodiment of the invention the present compounds are used for the preparation of a pharmaceutical composition for the delaying or prevention of the progression from non-insulin requiring type 2 diabetes to insulin requiring type 2 diabetes.

In a further embodiment of the invention the present compounds are used for the preparation of a pharmaceutical composition for the treatment of type 1 diabetes. Such treatment is normally accompanied by insulin therapy.

In yet a further embodiment of the invention the present compounds are used for the preparation of a pharmaceutical composition for the treatment of obesity.

In still a further embodiment of the invention the present compounds are used for the preparation of a pharmaceutical composition for the treatment of disorders of the lipid metabolism.

In still another embodiment of the invention the present compounds are used for the preparation of a pharmaceutical composition for the treatment of an appetite regulation or energy expenditure disorder.

In a further embodiment of the invention, treatment of a patient with the present compounds is combined with diet and/or exercise.

In a further aspect of the invention the present compounds are administered in combination with one or more further active substances in any suitable ratios. Such further active substances may eg be selected from antidiabetics, antiobesity agents, antihypertensive agents, agents for the treatment of complications resulting from or associated with diabetes and agents for the treatment of complications and disorders resulting from or associated with obesity.

Thus, in a further embodiment of the invention the present compounds may be administered in combination with one or more antidiabetics.

Suitable antidiabetic agents include insulin, insulin analogues and derivatives such as those disclosed in EP 792 290 (Novo Nordisk A/S), eg N^{εB29}-tetradecanoyl des (B30) human insulin, EP 214 826 and EP 705 275 (Novo Nordisk A/S), eg Asp^{B28} human insulin, U.S. Pat. No. 5,504,188 (Eli Lilly), eg Lys^{B28} Pro^{B29} human insulin, EP 368 187 (Aventis), eg Lantus®, which are all incorporated herein by reference, GLP-1 and GLP-1 derivatives such as those disclosed in WO 98/08871 (Novo Nordisk A/S), which is incorporated herein by reference, as well as orally active hypoglycemic agents.

The orally active hypoglycemic agents preferably comprise imidazolines, sulphonylureas, biguanides, meglitinides, oxadiazolidinediones, thiazolidinediones, insulin sensitizers, insulin secretagogues, such as glimepiride, α-glucosidase inhibitors, agents acting on the ATP-dependent potassium channel of the β-cells eg potassium channel openers such as those disclosed in WO 97/26265, WO 99/03861 and WO 00/37474 (Novo Nordisk A/S) which are incorporated herein by reference, or mitiglinide, or a potassium channel blocker, such as BTS-67582, nateglinide, glucagon antagonists such as those disclosed in WO 99/01423 and WO 00/39088 (Novo Nordisk A/S and Agouron Pharmaceuticals, Inc.), which are incorporated herein by reference, GLP-1 agonists such as those disclosed in WO 00/42026 (Novo Nordisk A/S and Agouron Pharmaceuticals, Inc.), which are incorporated herein by reference, GLP-1 antagonists, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, activators of glucokinase (GK) such as those disclosed in WO 00/58293, WO 01/44216, WO 01/83465, WO 01/83478, WO 01/85706, WO 01/85707, and WO 02/08209 (Hoffman-La Roche) or those disclosed in WO03/00262, WO 03/00267 and WO 03/15774 (AstraZeneca), which are incorporated herein by reference, GSK-3 (glycogen synthase kinase-3) inhibitors, compounds modifying the lipid metabolism such as antilipidemic agents such as HMG CoA inhibitors (statins), compounds lowering food intake, PPAR (peroxisome proliferator-activated receptor) ligands including the PPAR-alpha, PPAR-gamma and PPAR-delta substypes, and RXR (retinoid X receptor) agonists, such as ALRT-268, LG-1268 or LG-1069.

In one embodiment, the present compounds are administered in combination with insulin or an insulin analogue or derivative, such as N^{εB29}-tetradecanoyl des (B30) human insulin, Asp^{B28} human insulin, Lys^{B28} Pro^{B29} human insulin, Lantus®, or a mix-preparation comprising one or more of these.

In a further embodiment of the invention the present compounds are administered in combination with a sulphonylurea such as glibenclamide, glipizide, tolbautamide, chloropamidem, tolazamide, glimepride, glicazide and glyburide.

In another embodiment of the invention the present compounds are administered in combination with a biguanide eg metformin.

In yet another embodiment of the invention the present compounds are administered in combination with a meglitinide eg repaglinide or nateglinide.

In still another embodiment of the invention the present compounds are administered in combination with a thiazolidinedione insulin sensitizer eg troglitazone, ciglitazone, pioglitazone, rosiglitazone, isaglitazone, darglitazone, englitazone, CS-011/CI-1037 or T 174 or the compounds disclosed in WO 97/41097, WO 97/41119, WO 97/41120, WO 00/41121 and WO 98/45292 (Dr. Reddy's Research Foundation), which are incorporated herein by reference.

In still another embodiment of the invention the present compounds may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193 such as ragaglitazar (NN 622 or (−)DRF 2725) (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In a further embodiment of the invention the present compounds are administered in combination with an α-glucosidase inhibitor eg voglibose, emiglitate, miglitol or acarbose.

In another embodiment of the invention the present compounds are administered in combination with an agent acting on the ATP-dependent potassium channel of the β-cells eg tolbutamide, glibenclamide, glipizide, glicazide, BTS-67582 or repaglinide.

In yet another embodiment of the invention the present compounds may be administered in combination with nateglinide.

In still another embodiment of the invention the present compounds are administered in combination with an antilipidemic agent or antihyperlipidemic agent eg cholestyramine, colestipol, clofibrate, gemfibrozil, lovastatin, pravastatin, simvastatin, pitavastatin, rosuvastatin, probucol, dextrothyroxine, fenofibrate or atorvastin.

In still another embodiment of the invention the present compounds are administered in combination with compounds lowering food intake.

In another embodiment of the invention, the present compounds are administered in combination with more than one of the above-mentioned compounds eg in combination with metformin and a sulphonylurea such as glyburide; a sulphonylurea and acarbose; nateglinide and metformin; repaglinide and metformin, acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In a further embodiment of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocytestimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, H3 histamine antagonists, opioid antagonists (such as naltrexone), exendin-4, GLP-1 and ciliary neurotrophic factor (such as axokine), cannaboid receptor antagonist for example CB-1 (such as rimonabant).

In another embodiment the antiobesity agent is dexamphetamine or amphetamine.

In another embodiment the antiobesity agent is leptin.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

In still another embodiment the antiobesity agent is phendimetrazine, diethylpropion, fluoxetine, bupropion, topiramate or ecopipam.

Furthermore, the present compounds may be administered in combination with one or more antihypertensive agents. Examples of antihypertensive agents are 1-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

The compounds of the present invention may be administered in combination with FAS inhibitors.

The compounds of the present invention may also be administered in combination with chemical uncouplers, hormone sensitive lipase inhibitors, imidazolines, 11-β-hydroxysteroid dehydrogenase inhibitors, lipoprotein lipase activatore, AMPK activators, immunosuppresive drugs, nicotinamide, ASIS, anti-androgens or carboxypeptidase inhibitors.

It should be understood that any suitable combination of the compounds according to the invention with diet and/or exercise, one or more of the above-mentioned compounds and optionally one or more other active substances are considered to be within the scope of the present invention.

Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typical doses are in the order of about half the dose employed for oral administration.

The compounds of this invention are generally utilized as the free substance or as a pharmaceutically acceptable salt thereof. One example is a base addition salt of a compound having the utility of a free acid. When a compound of the formula (I) contains a free acid such salts are prepared in a conventional manner by treating a solution or suspension of a free acid of the formula (I) with a chemical equivalent of a pharmaceutically acceptable base. Representative examples are mentioned above.

For parenteral administration, solutions of the novel compounds of the formula (I) in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

A typical tablet that may be prepared by conventional tabletting techniques may contain:

Core:

| | | |
|---|---|---|
| Active compound (as free compound or salt thereof) | 5.0 | mg |
| Lactosum Ph. Eur. | 67.8 | mg |
| Cellulose, microcryst. (Avicel) | 31.4 | mg |
| Amberlite ® IRP88* | 1.0 | mg |

| | |
|---|---|
| Magnesii stearas Ph. Eur. | q.s. |

Coating:

| | | |
|---|---|---|
| Hydroxypropyl methylcellulose | approx. 9 | mg |
| Mywacett 9-40 T** | approx. 0.9 | mg |

*Polacrillin potassium NF, tablet disintegrant, Rohm and Haas.
**Acylated monoglyceride used as plasticizer for film coating.

If desired, the pharmaceutical composition of the invention may comprise the compound of the formula (I) in combination with further pharmacologically active substances such as those described in the foregoing.

### EXAMPLES

HPLC-MS (Method A)

The following instrumentation is used:

Hewlett Packard series 1100 G1312A Bin Pump

Hewlett Packard series 1100 Column compartment

Hewlett Packard series 1100 G13 15A DAD diode array detector

Hewlett Packard series 1100 MSD

The instrument is controlled by HP Chemstation software.

The HPLC pump is connected to two eluent reservoirs containing:

A: 0.01% TFA in water

B: 0.01% TFA in acetonitrile

The analysis is performed at 40° C. by injecting an appropriate volume of the sample (preferably 1 mL) onto the column, which is eluted with a gradient of acetonitrile.

The HPLC conditions, detector settings and mass spectrometer settings used are given in the following table.

| | |
|---|---|
| Column | Waters Xterra 100A MS C-18 3.5 μm, 3.0 mm × 50 mm |
| Gradient | 10%-100% acetonitrile lineary during 7.5 min at 1.0 mL/min |
| Detection | UV: 210 nm (diode array) |
| MS | Ionisation mode: API-ES |
| | Scan 100-1000 amu step 0.1 amu |

General Procedure (A)
General Procedure (A) for the Solution Phase Synthesis of Compounds of the General Formula (I₁) 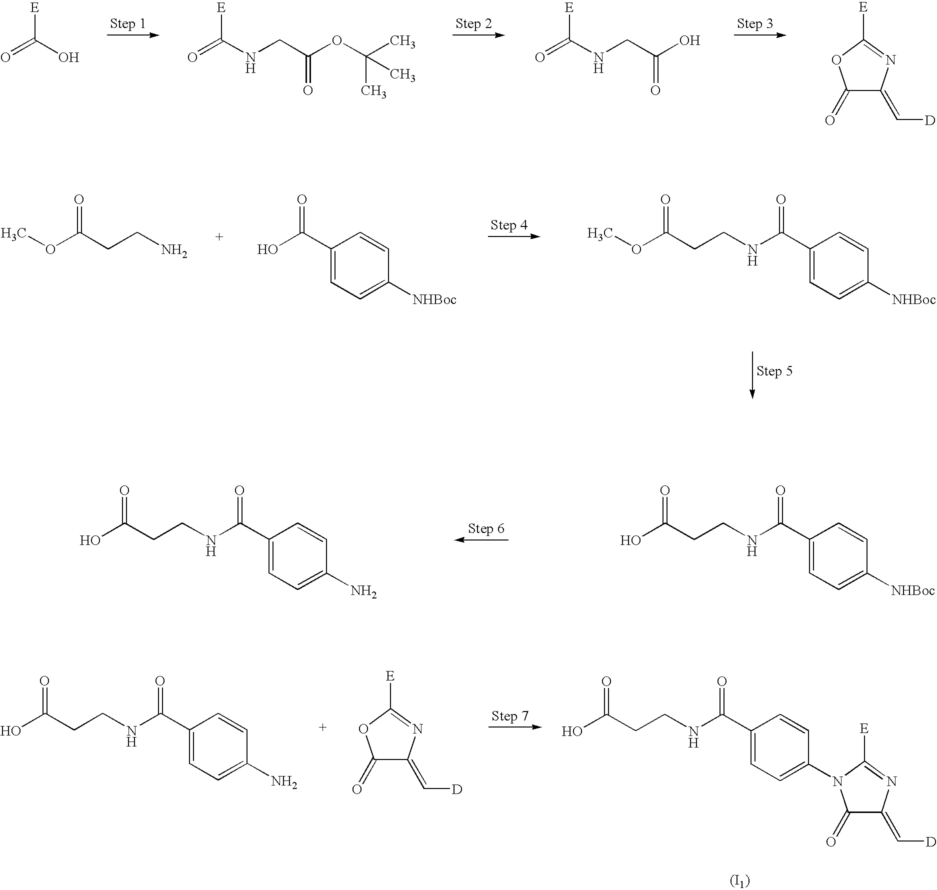
wherein D and E are as defined above
Steps 1 and 2:

These steps are simple amide coupling and deprotection steps to obtain substituted N-aroylglycines. These steps are well known to those skilled in the art.

Step 3: 2-Aryl-4-arylidene-oxazol-5-one formation

The formation of 2-aryl-4-arylidene-oxazol-5-one from the N-aroylglycine obtained in the previous steps is normally performed by treating the N-aroylglycine with substituted arylaldehydes in the presence of acetic anhydride and sodium acetate (see eg. Suzuki et al., J. Org Chem., 1983, 48, 4769-71, Sengupta and Gupta, J. Indian, Chem. Soc., 1984, 61, 724-6). Alternatively the N-aroylglycine is activated (ethyl chloroformate, triethylamine) and subsequently treated with arylideneanilines (see e.g. Kumar and Mukerjee, J. Indian Chem. Soc., 1981, 20B, 416-8)

Steps 4, 5 and 6: Preparation of 3-(4-aminobenzoyl)propionic acid

The reactions are generally is known and similar steps have been described in eg. WO 0069810. The steps consist of coupling of (N-protected) 4-aminobenzoic acid with 3-aminopropionic acid ester, followed by deprotection of the ester and amino groups. The protecting scheme can be varied well known to those skilled in the art.

Step 7: Condensation of the intermediates obtained in steps 3 and 6

This step is in principle a condensation of an aniline (obtained in step 6) with the oxazol-5-one obtained in step 3 to give an imidazol-5-one. This reaction is well known in the literature and is normally performed by heating the reactants in a mixture of acetic acid and sodium acetate (Habib et al., J. Prakt. Chem., 1986, 328, 295-300), heating the reactants in pyridine (Mathur and Sahay, J. Indian Chem. Soc., 1990, 67, 856-8), or heating the neat reactants (Bhatt et al., Indian J. Chem., 1999, 38B, 628-31).

The general procedure (A) is further illustrated in the following example:

### Example 1

### General procedure (A)

### 3-{4-[5-Oxo-4-(4-trifluoromethoxybenzylidene)-2-(4-trifluoromethoxyphenyl)-4,5-dihydroimidazol-1-yl]benzoylamino}propionic acid

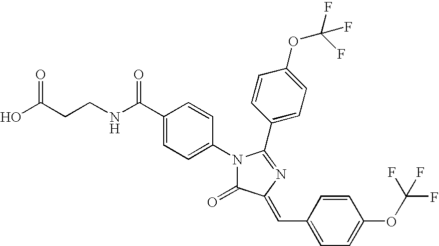 Reaction scheme:

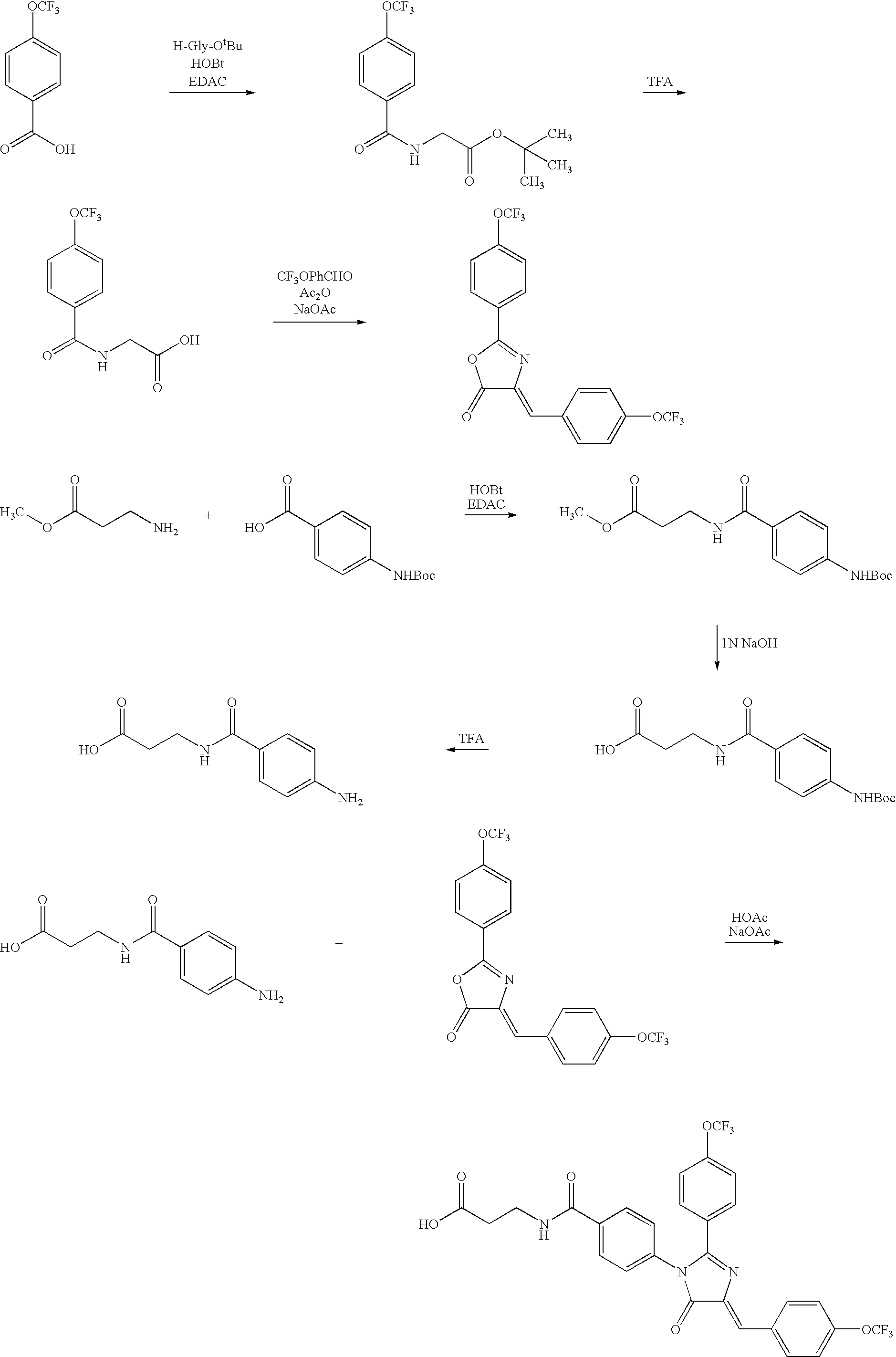

### Preparation of the intermediate 4-(4-Trifluoromethoxybenzylidene)-2-(4-trifluoromethoxyphenyl)-4H-oxazol-5-one

4-Trifluoromethoxybenzoic acid (5.50 g, 26.7 mmol) was dissolved in DMF (75 ml) and 1-hydroxybenzotriazol (3.96 g, 29.4 mmol), N-methylmopholine (5.87 ml, 53.4 mmol), and EDAC (5.63 g, 29.4 mmol) were added and the mixture was stirred at room temperature for 1 hour. Glycine tert-butyl ester hydrochloride (4.92 g, 29.4 mmol) was added and the mixture was stirred at room temperature for 16 hours. The mixture was concentrated in vacuo and the residue was partitioned between ethyl acetate (100 ml) and 1 N aqueous sodium hydroxide (100 ml). The organic phase was washed with 1 N aqueous sodium hydroxide (100 ml), dried (MgSO₄) and concentrated in vacuo to afford 5.97 g (70%) of (4-trifluoromethoxybenzoylamino)acetic acid tert-butyl ester as a solid.

¹H-NMR (300 MHz, CDCl₃): δ=1.52 (9H, s), 4.14 (2H, d), 6.83 (1H, t), 7.25 (2H, d), 7.88 (2H, d).

A mixture consisting of (4-trifluoromethoxybenzoylamino)acetic acid tert-butyl ester (5.63 g, 17.6 mmol), dichloromethane (35 ml) and trifluoroacetic acid (35 ml) was stirred at room temperature for 3 days. Concentration in vacuo afforded a quantitative yield of (4-trifluoromethoxybenzoylamino)acetic acid.

¹H-NMR (300 MHz, DMSO-d₆): δ=3.97 (2H, d), 7.48 (2H, d), 8.02 (2H, d), 9.00 (1H, t).

A mixture of (4-trifluoromethoxybenzoylamino)acetic acid (2.50 g, 9.50 mmol), 4-trifluoromethoxybenzaldehyde (2.05 ml, 14.3 mmol), anhydrous sodium acetate (2.34 g) and acetic anhydride (15 ml) was stirred vigorously at room temperature for 2 days. The mixture was poured into water (150 ml), stirred, and extracted with ethyl acetate (2×100 ml). he combined organic phases were dried (MgSO4) and concentrated in vacuo. The residue was washed with pentane (approx 10 ml) to afford 1.51 g (38%) of 4-(4-trifluoromethoxybenzylidene)-2-(4-trifluoromethoxyphenyl)-4H-oxazol-5-one as a solid.

¹H-NMR (300 MHz, DMSO-d₆): δ=7.42 (1H, s), 7.45 (2H, d), 7.58 (2H, d), 8.25 (2H, d), 8.43 (2H, d).

Preparation of the intermediate 3-(4-aminobenzoylamino)propionic acid trifluoroacetate:

4-(tert-Butoxycarbonylamino)benzoic acid (5.0 g, 21.1 mmol) was dissolved in DMF (75 ml) and 1-hydroxybenzotriazol (3.13 g, 23.2 mmol), 3-ethyl-1-(3-dimethylaminopropyl)carbodiimide hydrochloride (4.44 g, 23.2 mmol), and n-methylmorpholine (4.63 ml, 42.1 mmol) were added and the mixture was stirred at room temperature for 1 hour. Beta-alanine methyl ester hydrochloride (3.24 g, 23.2 mmol) was added and the resulting mixture was stirred at room temperature for 16 hours. The mixture was concentrated in vacuo and the residue was partitioned between ethyl acetate (200 ml) and 1 N aqueous sodium hydroxide (200 ml). The organic phase was washed with 1 N aqueous sodium hydroxide (100 ml), dried (MgSO₄) and concentrated in vacuo to afford 6.94 g (100%) of 3-[4-(tert-butoxycarbonylamino)benzoylamino]propionic acid methyl ester as a solid.

¹H-NMR (300 MHz, DMSO-d₆): δ=1.50 (9H, s), 2.60 (2H, t), 3.47 (2H, q), 3.61 (3H, s), 7.51 (2H, d), 7.75 (2H, d), 8.40 (1H, t), 9.60 (1H, s).

HPLC-MS (Method (A)): m/z: 323 (M+1); Rt: 3.04 min.

3-[4-(tert-Butoxycarbonylamino)benzoylamino]propionic acid methyl ester (6.28 g, 19.5 mmol) was dissolved in methanol (150 ml) and 1 N aqueous sodium hydroxide (25 ml) was added and the resulting mixture was stirred at room temperature for 16 hours. The mixture was concentrated in vacuo (120 mBar, 40° C.) and the residue was diluted with water (150 ml). 1 N hydrochloric acid (50 ml) was added. The mixture was filtered and the solid was isolated by filtration, washed with water and air-dried. This afforded 5.76 g (96%) of 3-[4-(tertbutoxycarbonylamino)benzoylamino]propionic acid.

HPLC-MS (Method (A)): m/z: 309 (M+1); Rt: 2.63 min.

3-[4-(tert-Butoxycarbonylamino)benzoylamino]propionic acid (5.39 g, 17.5 mmol) was suspended in dichloromethane (50 ml) and trifluoroacetic acid (50 ml) was added and the mixture was stirred at room temperature for 16 hours, concentrated in vacuo and stripped twice with toluene. This afforded a quantitative yield of 3-(4-aminobenzoylamino)propionic acid trifluoroacetate, used in the next step without characterisation.

Preparation of the Title Compound:

A mixture of 3-(4-aminobenzoylamino)propionic acid trifluoroacetate (0.427 g, 1.33 mmol), 4-(4-trifluoromethoxybenzylidene)-2-(4-trifluoromethoxyphenyl)-4H-oxazol-5-one (0.553 g, 1.33 mmol), anhydrous sodium acetate (0.436 g, 5.32 mmol) and acetic acid glacial (7 ml) was refluxed for 4 hours and stirred at room temperature for 16 hours. The mixture was filtered and the solid was washed with a little acetic acid and water. Drying in vacuo at 40° C. for 16 hours afforded 0.35 g (43%) of the title compound.

¹H-NMR (300 MHz, DMSO-d₆): δ=2.55 (t, below DMSO), 3.49 (2H, q), 7.4-7.5 (5H, m), 7.53 (2H, d), 7.67 (2H, d), 7.93 (2H, d), 8.50 (2H, d), 8.67 (1H, t), 12.3 (1H, bs).

HPLC-MS (Method (A)): m/z: 608 (M+1); Rt: 4.95 min.

### Example 2

### General Procedure (A)

### 3-{4-[2-(4-Cyclohexylphenyl)-4-(3,5-dichlorobenzylidene)-5-oxo-4,5-dihydroimidazol-1-yl]-benzoylamino}propionic acid

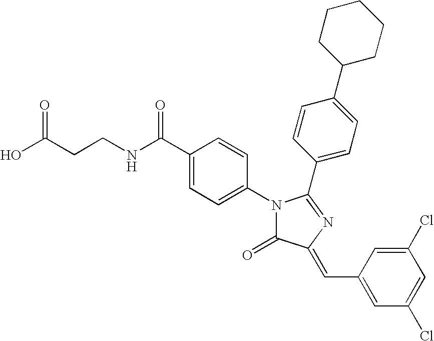

HPLC-MS (Method (A)): m/z: 590 (M+1); Rt: 6.07 min.

### Example 3

### General Procedure (A)

### 3-{4-[2-Biphenyl-4-yl-5-oxo-4-(4-trifluoromethoxybenzylidene)-4,5-dihydroimidazol-1-yl]benzoylamino}propionic acid

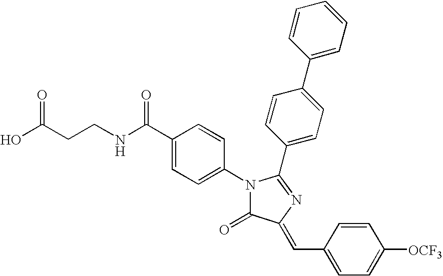

HPLC-MS (Method (A)): m/z: 600 (M+1); Rt: 5.51 min.

### Example 4

### General Procedure (A)

### 3-{4-[2-(4-Cyclohexylphenyl)-5-oxo-4-(4-trifluoromethoxybenzylidene)-4,5-dihydroimidazol-1-yl]benzoylamino}propionic acid

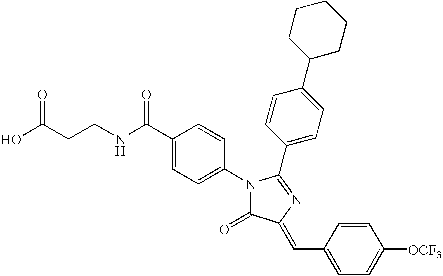

HPLC-MS (Method (A)): m/z: 606 (M+1); Rt: 5.74 min.

General Procedure (B)

General procedure (B) for solid phase synthesis of compounds of the general formula (I₂): 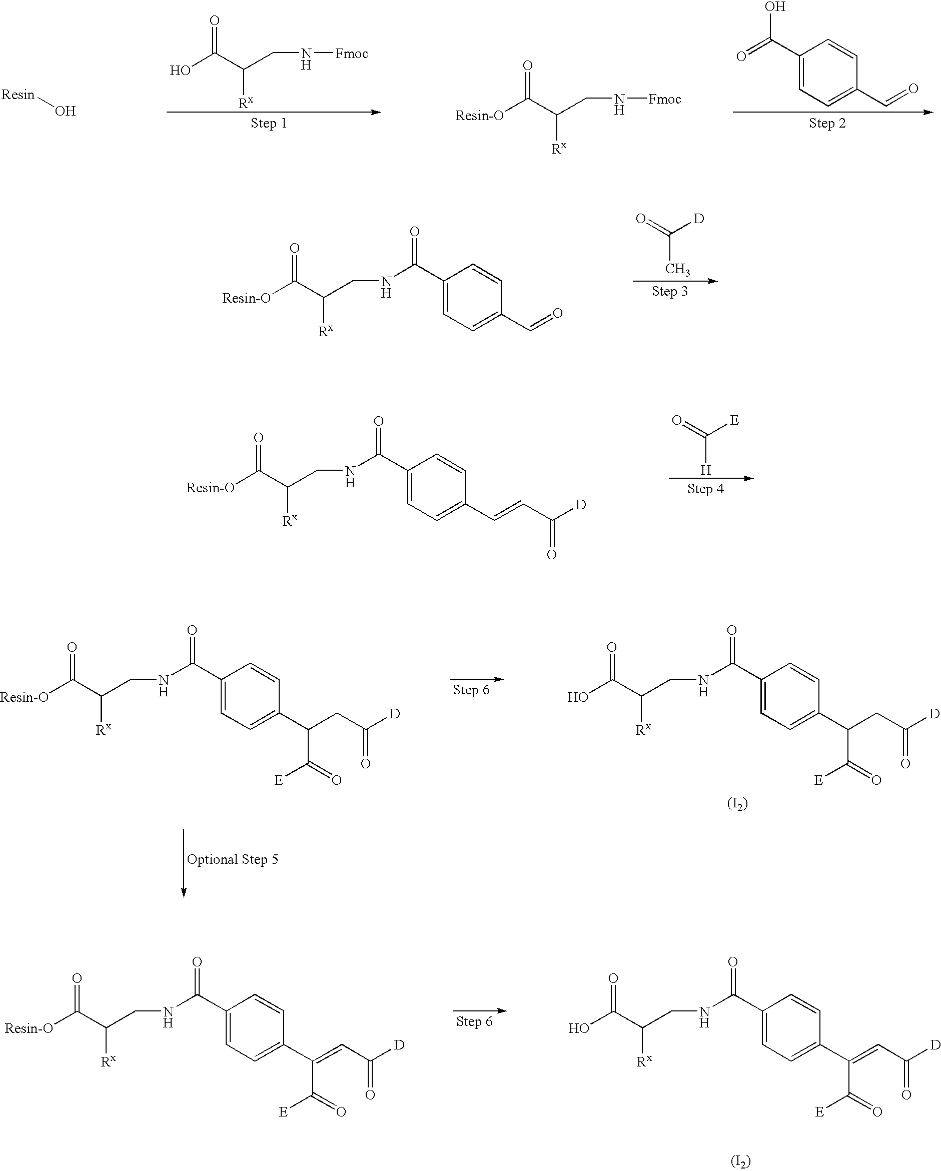
wherein R^{x} is either H or OH according to formula (I), D and E are as defined for formula (I), and Resin is a polystyrene resin loaded with a Wang-linker.
Step 1:

This reaction is known (Wang S. J., J. Am. Chem. Soc. 95, 1328, 1973) and is generally performed by stirring polystyrene resin loaded with a linker such as the Wang linker with a 4-10 molar excess of N-fluorenylmethylcarbonyl (Fmoc)-protected amino acid activated with a 2-5 molar excess of diisopropylcarbodiimide, dicyclohexylcarbodiimide or 1-[3-(dimethylamino)propyl]-3-ethylcarbodiimide hydrochloride in the presence of a catalyst such as N,N-4-dimethylaminopyridine. The esterfication is carried out in solvents such as THF, dioxane, toluene, DCM, DMF, NMP or a mixture of two or more of these. The reactions are performed between 0° C. and 80° C., preferably between 20° C. to 40° C. When the esterification is complete excess of reagent is removed by filtration. The resin is successively washed with the solvent used in the reaction, followed by washing with methanol. The resin bound product can be further dried and analyzed.

Step 2:

N-Fluorenylmethylcarbonyl protecting group is removed by treating the resin bound derivative with a 20%-50% solution of a secondary amine such as piperidine in a polar solvent such as DMF or NMP (Carpino L., Han G., J. Org. Chem. 37, 3404, 1972). The reaction is performed between 20° C. to 180° C., preferably between 20° C. to 40° C. When the reaction is complete excess of reagent is removed by filtration. The resin is successively washed with solvent used in the reaction. The resulting resin bound intermediate is acylated with acid. The acylation is known (The combinatorial index, Ed. Bunin B. A., 1998, Acedemic press, p. 78) and is generally performed by stirring the resin bound intermediate with a 2-5 molar excess of acid activated with a 2-5 molar excess of of diisopropyl-carbodiimide, dicyclohexylcarbodiimide or 1-[3-(dimethylamino)propyl]-3-ethylcarbodiimide hydrochloride in the presence of a side reaction inhibitor such as N-hydroxybenzotriazole. The acylation is carried out in a solvent such as THF, dioxane, toluene, DCM, DMF, NMP or a mixture of two or more of these. The reactions are performed between 0° C. to 80° C., preferably between 20° C. to 40° C. When the esterification is complete excess of reagent is removed by filtration. The resin is successively washed with the solvent used in the reaction, followed by washing with methanol. The resin bound product can be further dried and analyzed.

Step 3:

This reaction is a modification of previously described procedures for aldol condensation on solid support (Sensfuss U. Tetrahedron Letters 44 2371-2374 (2003). The reaction is carried out by reacting polystyrene-linked benzaldehydes with methyl ketones in presence of cobalt(II) or zinc acetate 2,2′-bipyridine complexes and an amidine base at elevated temperature to give resin-bound (E)-enones. The reaction is carried out in a polar aprotic solvent like DMF or NMP. The reactions are performed 40° C. to 120° C. preferreably at 70° C.-80° C. When the aldol condensation is complete excess of reagent is removed by filtration. The resin is successively washed with the solvent used in the reaction, followed by washing with methanol. The resin bound product can be further dried and analyzed.

Step 4:

This reaction is known (Sadagopan S., Anuradha, K. Tetrahedron Letters. 43, 5181-5183, 2002). The addition of aldehydes to activated double bonds is generally carried out by stirring the aldehyde with a compound that contains an activated double bond such as a substituted propenone in the presence of a catalyst such as sodium or potassium cyanide or thiazolium salts such as 3,4-dimethyl-5-(2-hydroxyethyl)thiazolium iodide, 3-benzyl-5-(2-hydroxyethyl)-4-methyl-1,3-thiazolium chloride, 3-ethyl-5-(2-hydroxyethyl)-4-methyl-1,3-thiazolium bromide or vitamin B₁. When thiazolium salts are used as catalyst, a non-nucleophilic amine base such as triethyl amine, N,N-diisopropylethylamine or DBU is added. The addition is carried out in a solvent such as dioxane, DMSO, NMP or DMF or a mixture of two or more of these. The reactions are performed between 50° C. to 120° C., preferably between 50° C. to 80° C. When the reaction is complete, excess of reagent is removed by filtration. The resin is successively washed with the solvent used in the reaction, followed by washing with methanol. The resin bound product can be further dried and analyzed.

Step 5:

The reaction is performed using iodine and DBU in THF and is described in WO 03/048109, General procedure (C), step 4).

Step 6:

The reaction is known (The combinatorial index, Ed. Bunin B. A., 1998, Acedemic press, p. 21) and is generally performed by stirring the resin bound intermediate obtained in step 3 with a 50-95% solution of TFA. The final cleavage is carried out in a solvent such as THF, DCM, 1,2 dichloroethane, 1,3-dichloropropane, toluene or a mixture or more of these. The reactions are performed between 0° C. to 80° C., preferably between 20° C. to 40° C. When the reaction is complete the product is removed by filtration. The resin is successively washed with DCM. The product and washings are collected. The solvent is removed and the product is dried in vacuo.

The procedure is further illustrated in the following example.

### Example 5

### (General Procedure (B))₃-{4-[3-(4-Cyclohexylphenyl)-3-oxo-1-(4-trifluoromethoxybenzoyl)propyl]benzoylamino}propionic acid

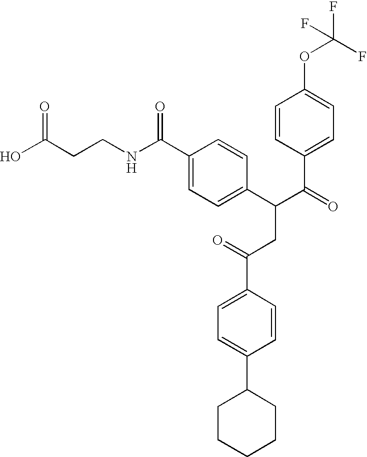

### Step 1 and Step 2: Resin bound 3-(4-formylbenzoylamino)propionic acid

3-(4-Formylbenzoylamino)propionic acid resin bound to a Wang resin (loading approximately 0.2-0.8 mmol/g) was synthesized according to the procedure described in WO 00/69810.

### Step 3: Preparation of resin bound 3-(4-(3-(4-cyclohexylphenyl)-3-oxopropenyl)benzoylamino)propionic acid

The above resin bound 3-(4-formylbenzoylamino)propionic acid (1 g resin) was suspended in DMF (20 mL) for 30 min and filtered. 4-cyclohexylacetophenone (4.05 g, 20 mmol) was dissolved in DMF (10 mL) and added to the resin. Zinc(II)acetate dihydrate (220 mg, 1 mmol) and 2,2′-bipyridine (156 mg, 1 mmol) was dissolved in DMF (10 mL) and added. DBU (2 mmol) was added and the suspension was shaken at 70° C. for 16 hours. The resin was isolated by filtration and washed with methanol (1×20 mL) and NMP (2×20 mL).

### Step 4 and Step 5 Preperation of 3-{4-[3-(4-cyclohexylphenyl)-3-oxo-1-(4-trifluoromethoxybenzoyl)propyl]benzoylamino}propionic acid

To the above resin bound 3-(4-(3-(4-cyclohexylphenyl)-3-oxopropenyl)benzoylamino)propionic acid was added 3,4-Dimethyl-5-(2-hydroxyethyl)thiazolium iodide (2.85 g, 10 mmol) was dissolved in NMP (20 mL). 4-(Trifluoromethoxy)benzaldehyde (3.8 g, 10 mmol) was added followed by DBU (10 mmol). The suspension was shaken at 70° C. for 16 hours. The resin was isolated by filtration and washed with methanol (1×20 mL), DCM containing 5% acetic acid (1×20 mL) followed by DCM (3×(20 mL). The resin bound 3-{4-[3-(4-Cyclohexylphenyl)-3-oxo-1-(4-trifluoromethoxybenzoyl)propyl]benzoylamino}propionic acid was treated with 50% TFA in DCM (20 mL) for 0.5 hour at 25° C. The mixture was filtered and the resin was washed with DCM (20 mL). The combined filtrates were concentrated in vacuo to afford an oil which was purified on silica gel column eluted with DCM/ethanol (95:5) to afford the title compound.

¹H NMR (CDCl₃): δ 1.15-1.50 (m, 5H), 1.70-1.92 (m, 5H), 2.55 (m, 1H), 2.67 (t, 2H), 3.30 (dd, 1H), 3.70 (q, 3H), 4.17 (dd, 1H), 5.30 (dd, 1H), 6.93 (t, 1H), 7.20 (d, 2H), 7.28 (d, 2H), 7.40 (d, 2H), 7.70 (d, 2H), 7.88 (d, 2H), 8.03 (d, 2H).

### Example 6

### (General procedure (B))Z-3-{4-[3-(4-Cyclohexylphenyl)-3-oxo-1-(4-trifluoromethoxybenzoyl)propenyl]benzoylamino}propionic acid

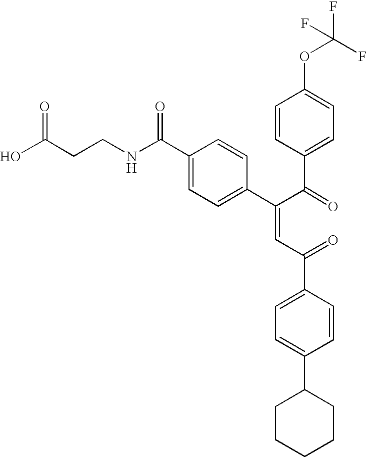

Iodine (153 mg, 0.6 mmol) was dissolved in THF (4 mL) and DBU (0.271 mL, 1.8 mmol.) was added. This solution was added to 3-{4-[3-(4-cyclohexylphenyl)-3-oxo-1-(4-trifluoromethoxybenzoyl)propyl]benzoylamino}propionic acid (300 mg, 0.5 mmol) and stirred at room temperature for 30 minutes. The solution was diluted with diethyl ether (30 mL) and washed with a sodium sulfite solution (2% in water, 2×30 mL). The organic phase was washed with 1 N hydrochloric acid (30 mL), dried (Na₂SO₄) and solvent removed by evaporation. A foam appeared which was redisolved in toluene (30 mL) and 4 drops of concentrated hydrochloric acid was added. The mixture was heated to reflux for 30 minutes, and evaporated give the title compound.

¹H NMR (CDCl₃): δ 1.10-1.50 (m, 5H), 1.60-1.95 (m, 5H), 2.55 (m, 1H), 2.67 (t, 2H), 7.08 (br s, 1H), 7.20 (d, 2H), 7.38 (d, 2H), 7.60 (d, 2H), 7.68 (s 1H), 7.78 (d, 2H), 7.90 (d, 2H), 7.95 (d, 2H).

General Procedure (C)

General procedure (C) for solid phase synthesis of compounds of the general formula (I₃ and I₄): 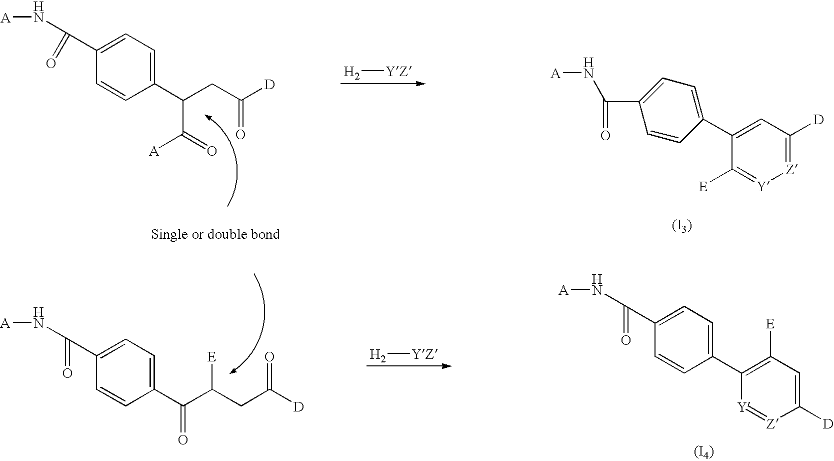
wherein X, D, E, m, n and A are as defined for formula (I), and Resin is a polystyrene resin loaded with a Wang-linker.

The indicated bonds are either single or double bonds (to give 5 and 6-membered heteroaromatic ring systems, respectively).

The reagent H₂—Y′Z′ is NH₂NH₂, H₂O, H₂S, NH₂R′ or any salt or hydrate thereof, —Y′=Z′- (or ═Y′-Z′=) is —N═N- (or ═N—N═), —O—, —S—, —NR′—, wherein R′ is hydrogen, lower alkyl, lower alkoxy, hydroxy, amino, lower alkylaryl, or aryl wherein the alkyl and aryl moieties are optionally substituted as defined above.

The reactions are known, see eg. J. March “Advanced Organic Chemistry”, 3^{rd} Edition, John Wiley and sons, 1985, p 791, and H. Stetter & H. Kuhlmann, Organic Reactions, 1991, 40, 407-496 and are generally performed by as described below.

When —Y′=Z′- is —NR′—: The reaction is generally performed in the presence of a NH₂R′ and a catalyst like a protic acid or Lewis acid. The reactions are performed between 50° C. and 150° C., in an organic solvent.

When —Y′=Z′- is —O—: The reaction is generally performed in the presence of a dehydrating reagent like (CH₃CO)₂O or BF₃ in the presence of an acid, such as H₂SO₄, H₃PO₄, HCl, TsOH or by acid alone in a non aqueous environment.

When —Y=Z- is —S—: The reaction is generally performed with P₂S₅, P₄S₁₀ or Lawesons reagent or by H₂S in the precence of an acid like HCl. As these reagents also will react with the other carbonyl groups in the starting materials, a slightly altered synthesis strategy may be used: Protected benzoic acids as starting materials, eg:

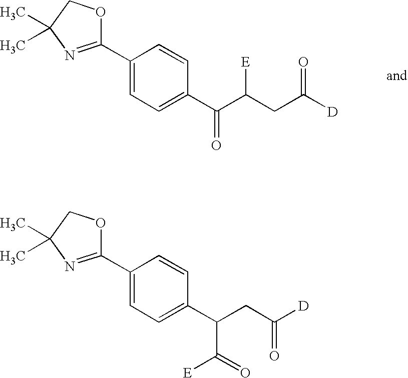

When —Y=Z- is —N═N- (or ═N—N═): The reaction is generally performed using hydrazine in a organic solvent like ethanol, DMF, NMP, DMSO or the like.

Preparation of the starting materials for this procedure is either described herein or in WO2003048109.

The procedure is further illustrated in the following examples.

### Example 7

### General Procedure (C)

### 3-{4-[6-(4-Cyclohexylphenyl)-3-(4-trifluoromethoxyphenyl)pyridazin-4-yl]benzoylamino}propionic acid

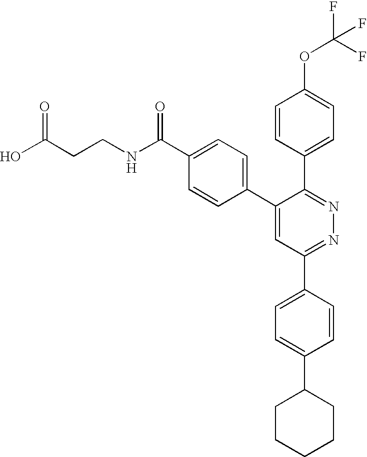

Z-3-{4-[3-(4-Cyclohexyl phenyl)-3-oxo-1-(4-trifluoromethoxybenzoyl)propenyl]benzoylamino}-propionic acid (100 mg, 0.17 mmol) was dissolved in ethanol (2 mL), hydrazine hydrate (80%) (0.1 ml, 3.4 mmol) was added and the mixture was stirren at room temperature overnight.

The reaction mixture was diluted with dichloromethane (30 mL) and washed with water (2×30 mL). The organic phase was dried (Na2SO4) and solvent removed in vacuo to afford an oil which was purified on silica gel column eluted with dichloromethane/ethanol/acetic acid (90:9:1) to afford the title compound.

HPLC-MS (Method A): m/z=590 (M+1); Rt=5.022 min.

### Example 8

### General Procedure (C)

### 3-{4-[4-(4-Cyclohexylphenyl)-6-(4-trifluoromethoxyphenyl)pyridazin-3-yl]benzoylamino}propionic acid

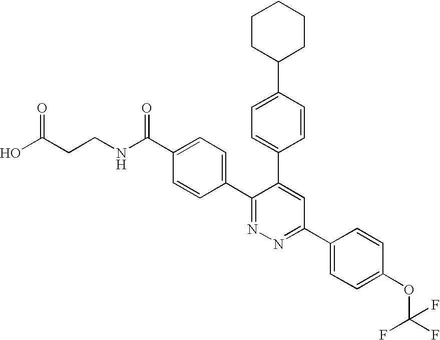 Step 1: Z-3-{4-[2-(4-Cyclohexyl phenyl)-4-oxo-4-(4-trifluoromethoxyphenyl)-but-2-enoyl]benzoylamino}propionic acid

This compound was prepared as described in WO2003048109.

### Step 2: 3-{4-[4-(4-Cyclohexylphenyl)-6-(4-trifluoromethoxyphenyl)pyridazin-3-yl]benzoylamino}propionic acid

Z-3-{4-[2-(4-Cyclohexyl phenyl)-4-oxo-4-(4-trifluoromethoxyphenyl)-but-2-enoyl]-benzoylamino}propionic acid (53 mg, 0.090 mmol) was dissolved in ethanol (3,5 mL). Hydrazine hydrate was added (0.18 mL) and the mixture was stirred overnight at room temperature. Toluene (50 mL) is added and solvent removed in vacuo crude product was purified on HPLC (RP-18) (Oprenset af HDEM HPLC oprensnings service, hendes metode kenderjeg ikke) to give the title compound.

¹H NMR (CDCl3): δ 1.36-1.47 (m, 5H), 1.82-1.93 (m, 5H), 2.72 (t, 2H), 3.73 (q, 2H), 7.15 (t, 1H), 7.20 (d, 2H), 7.40 (d, 2H), 7.57 (d, 2H), 7.71 (d, 2H), 7.87 (s, 1H), 8.21 (d, 2H); HPLC-MS (Method A): m/z=590 (M+1); Rt=5.427 min.

### Example 9

### General Procedure (C)

### 3-{4-[6-(3,5-Bis-trifluoromethyl phenyl)-4-(4-cyclohexyl-phenyl)-pyridazin-3-yl]-benzoylamino}-propionic acid

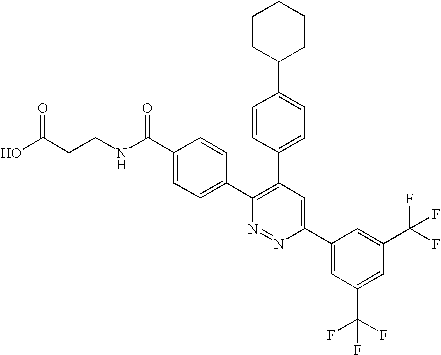

This compound could be prepared similar to described above (example 8)

HPLC-MS (Method A): m/z=643 (M+1); Rt=5.69 min.

### Example 10

### General Procedure (C)

### 3-{4-[3-(4-Cyclohexyl-phenyl)-5-(4-trifluoromethylsulfanyl-phenyl)-1H-pyrrol-2-yl]-benzoylamino}-propionic acid

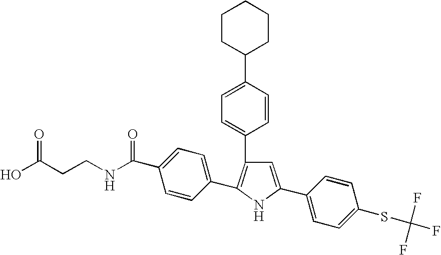

### Step 1: 3-{4-[2-(4-Cyclohexylphenyl)-4-oxo-4-(4-trifluoromethylsulfanylphenyl)butyryl]benzoylamino}propionic acid

This compound was prepared as described in WO2003048109.

### Step 2: 3-{4-[3-(4-Cyclohexylphenyl)-5-(4-trifluoromethylsulfanyl phenyl)-1H-pyrrol-2-yl]benzoylamino}propionic acid

3-{4-[2-(4-Cyclohexylphenyl)-4-oxo-4-(4-trifluoromethylsulfanyl phenyl)butyryl]benzoylamino}-propionic acid (0.1 g, 0.163 mmol) was dissolved in acetic acid (2 mL), ammonium acetate (1 g, 12.97 mmol) was added and the mixture was heated to 110° C. for 1.5 hours, cooled to room temperature, diluted with ethyl acetate (5 mL). The organic phase was washed with water (2×5 ml) followed by washing with saturated aqueous sodium chloride. The organic phase was dried (Na₂SO₄) and the solvent removed in vacuo to give 3-{4-[3-(4-cyclohexylphenyl)-5-(4-trifluoromethylsulfanylphenyl)-1H-pyrrol-2-yl]benzoylamino}propionic acid.

¹H NMR (CDCl₃): δ 1.11-1.45 (m, 5H), 1.66-1.90 (m, 5H), 2.05 (s, 1H), 2.44 (br s, 2H), 3.52 (brs, 2H), 6.63 (brs, 1H), 6.93 (s, 1H), 7.00 (d, 2H), 7.12 (d, 2H), 7.21 (d, 2H), 7.38 (d, 2H), 7.51 (d, 2H); 7.61 (d, 2H), 9.80 (s, 1H), HPLC-MS (Method A): m/z=593 (M+1); Rₜ=6.249 min.

### Example 11

### 3-{4-[2-(4-Cyclohexylphenyl)-3-(4-trifluoromethoxybenzoyl)cyclopropyl]benzoylamino}propionic acid

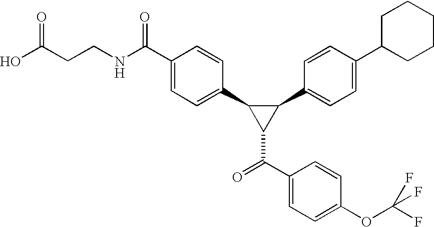

### Step 1: 4-Phenylsulfanylmethylbenzoic acid methylester

Methyl-4-(bromomethyl)benzoate (5.05 g, 22 mmol) was dissolved in dry DMF (100 mL) the solution was cooled in an ice bath under nitrogen. Potassium carbonate (6.09 g, 44,1 mmol) was added followed by thiophenol (2.3 mL, 22 mmol) while cooling was maintained. The mixture was stirred under nitrogen for 2 hours and poured into water (400 mL). The crude product precipitated and was collected by filtration. The crude product could be recrystalized from methanol to give 4.87 g (86%) of 4-phenylsulfanylmethylbenzoic acid methylester.

HPLC-MS (Method A): m/z=259 (M+1); Rₜ=4.67 min.

### Step 2: 4-Benzenesulfinylmethylbenzoic acid methyl ester

4-Phenylsulfanylmethylbenzoic acid methylester (4.87 g, 18,9 mmol) was dissolved in dichloromethane (100 mL). 3-Chlorobenzenecarboperoxoic acid (4.22 g) was added slowly over 15 minutes. The mixture was stirred for 1.5 hour at room temperature. The mixture was washed with a 2% solution of Na₂S₂O₅ in water (100 mL), followed by wash with saturated aqueous sodium carbonate (100 mL). The organic phase was dried (Na₂SO₄) and the solvent removed in vacuo to afford 4-benzenesulfinylmethylbenzoic acid methyl ester.

HPLC-MS (Method A): m/z=275 (M+1); Rt=3.1 min.

### Step 3: 4-[2-(4-Cyclohexylphenyl)-3-(4-trifluoromethoxybenzoyl)cyclopropyl]benzoic acid methyl ester

4-Benzenesulfinylmethylbenzoic acid methylester (0.82 g, 3.01 mmol) and 3-(4-Cyclohexylphenyl)-1-(4-trifluoromethoxyphenyl)propenone (1.13 g, 3.01 mmol, prepared as described in WO2003048109) were dissolved in dry pyridine (25 mL) and the pyridine subsequently removed by evaporation, followed by an additional evaporation from toluene (25 mL). The mixture was dissolved in dry DMF (10 mL) 60% suspension of sodium hydride in mineral oil (0,143 g, 3.6 mmol) was added and the suspension was stirred under nitrogen for 1 hour. The reaction was quenched with acetic acid (2 mL) stirred for 5 minutes and heated to 120° C. for 30 minutes. After cooling, the mixture was partitioned between diethyl ether (2×50 mL) and water (2×50 mL). The combined organic phases were was dried (Na₂SO₄) and solvent removed in vacuo. The crude product was purified on silica gel column eluted with ethylacetate/heptane (1:6) to afford 4-[2-(4-cyclohexylphenyl)-3-(4-trifluoromethoxybenzoyl)cyclopropyl]benzoic acid methyl ester as a mixture of stereoisomers.

### Step 4: 4-[2-(4-Cyclohexylphenyl)-3-(4-trifluoromethoxybenzoyl)cyclopropyl]benzoic acid

4-[2-(4-Cyclohexylphenyl)-3-(4-trifluoromethoxybenzoyl)cyclopropyl]benzoic acid methyl ester (0.6 g, 1.15 mmol) was dissolved in THF (10 mL), Potassium trimethylsilanolate (0.44 g, 3.44 mmol) was added and the mixture was stirred overnight under nitrogen at room temperature. The reaction was quenched with acetic acid (0.59 mL), separated between ethyl acetate (50 mL) and water (50 mL). The organic phase was dried (Na₂SO₄) and solvent removed in vacuo. The crude product was crystallized from toluene/heptane (20/80) to give the title compound (0.2 g, 35%) as one stereoisomer. HPLC-MS (Method A): m/z=509 (M+1); Rₜ=6.31 min

### Step 5: 3-{4-[2-(4-Cyclohexylphenyl)-3-(4-trifluoromethoxybenzoyl)cycloproyl]benzoylamino}propionic acid methyl ester

4-[2-(4-Cyclohexylphenyl)-3-(4-trifluoromethoxybenzoyl)cyclopropyl]benzoic acid (0.20 g, 0.40 mmol) was dissolved in DMF (3 mL). 1-(3-Dimethylaminopropyl)-3-ethylcarbodiimide hydrochloride (72 mg, 0.4 mmol) and 1-hydroxybenzotriazole hydrate (62 mg, 0.48 mmol) were added. The mixture was stirred for 1.5 hour. Beta-alanine methyl ester hydrochloride (83 mg, 0.60 mmol) and diisopropylethylamine (0.16 mL, 0.92 mmol) were added and the mixture was stirred overnight at room temperature. The mixture was partitioned between ethyl acetate (50 mL) and 1 N hydrochloric acid (2×50 mL). The organic phase was dried (Na₂SO₄) and the solvent removed in vacuo to give 3-{4-[2-(4-cyclohexylphenyl)-3-(4-trifluoromethoxybenzoyl)cyclopropyl]benzoylamino}propionic acid methyl ester (0.23 g, 97%). HPLC-MS (Method A): m/z=594 (M+1); Rₜ=6.07 min

### Step 6: 3-{4-[2-(4-Cyclohexylphenyl)-3-(4-trifluoromethoxybenzoyl)cycloproyl]benzoylamino}propionic acid

3-{4-[2-(4-Cyclohexylphenyl)-3-(4-trifluoromethoxybenzoyl)cyclopropyl]benzoylamino}propionic acid methyl ester (0.23 g, 0.389 mmol) was dissolved in ethanol (4 mL). 4 N aqueous sodium hydroxide (1 mL) was added and the mixture was stirred at room temperature overnight. The mixture was diluted with water (50 mL) and 1 N hydrochloric acid (4.5 mL). The crude product was isolated by filtration and further purified by silica gel column chromatography eluting with dichloromethane/methanol/acetic acid (96:3:1) to afford the title compound (53 mg, 24%).

¹H NMR (CDCl₃): δ 1.12-1.46 (m, 5H), 1.68-1.92 (m, 5H), 2.33-2.48 (m, 1H), 2.68 (t, 2H), 2.93 (d, 1H), 3.30 (d, 2H), 3.47 (T, 1H), 3.70 (q, 2H), 6.77 (t, 1H), 6.89 (d, 2H), 7.00 (d, 2H), 7.03 (d, 2H); 7.32 (d, 2H), 7.54 (d, 2H), 8.13 (d, 2H). HPLC-MS (Method A): m/z=580 (M+1); Rt=5.721 min.

### Example 12

### 3-{4-[5-(4-tert-Butylbenzoyl)-4-(4-cyclohexylphenyl)isoxazol-3-yl]benzoylamino}propionic acid

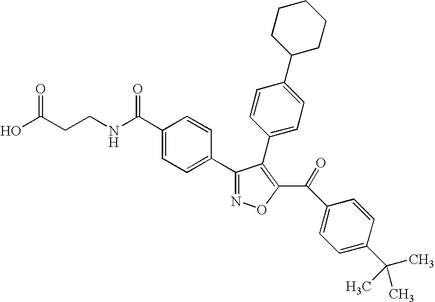 Reaction Scheme for Example 12:

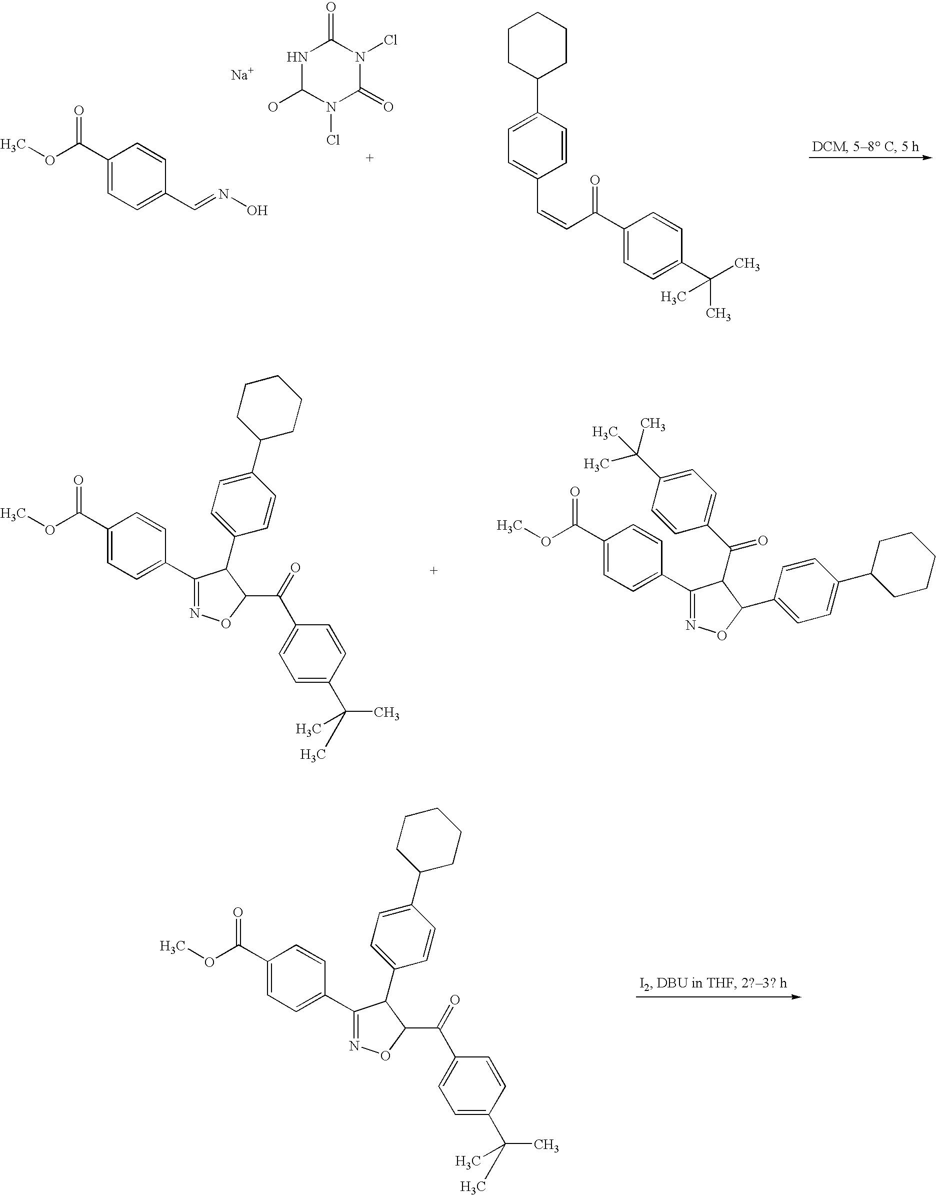

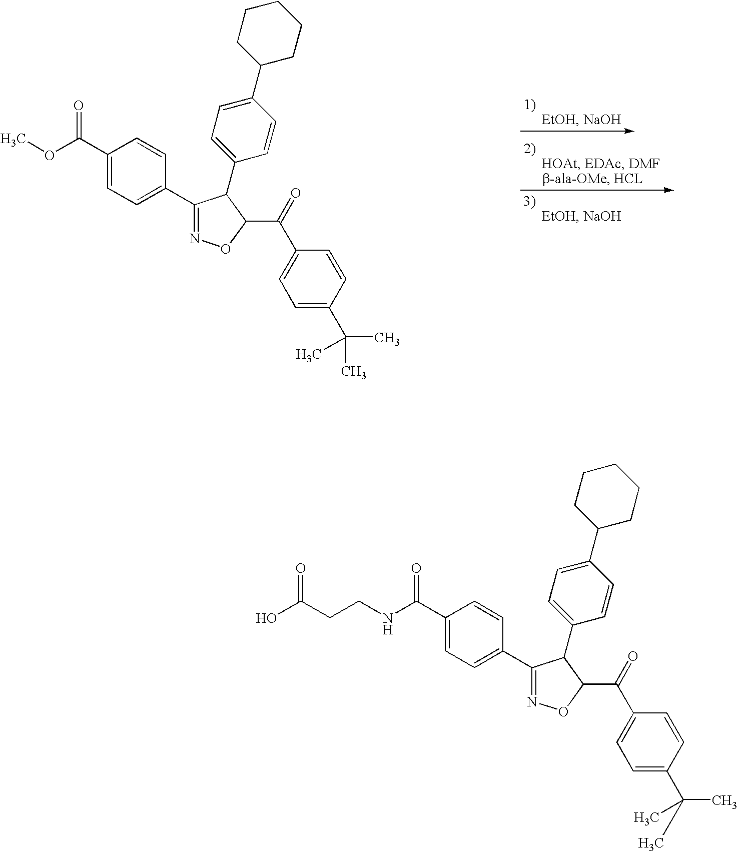

### 4-(Hydroxyiminomethyl)benzoic acid methyl ester

Methyl 4-formylbenzoate (2.0 g, 12.18 mmol) and hydroxylammoniumchloride (2.0 g, 28.8 mmol) were refluxed for one hour in a mixture of ethanol (99%, 20 mL) and pyridine (1 mL). The reaction mixture was concentrated in vacuo and the residue was washed with water (20 mL), filtered, and dried to give 2.1 g of 4-(hydroxyiminomethyl)benzoic acid methyl ester.

HPLC-MS (Method A): m/z=180 (M+1); Rt=2.36 min

### 1-(4-tert-Butylphenyl)-3-(4-cyclohexylphenyl)propenone

4-Cyclohexylbenzaldehyde (46.1 g, 0.245 mmol) and 4-tert-butylacetophenone (43.2 g, 0.245 mmol) were dissolved in ethanol (450 mL). Sodium hydroxide (9.8 g, 0.245 mmol) was dissolved in water (50 mL) added slowly to the reaction mixture. The mixture was stirred for 6 hours and filtered. The isolated solid was washed with water and dried in vacuo at 30° C. to give 67 g of crude product which was recrystallized from n-heptane to give 1-(4-tertbutylphenyl)-3-(4-cyclohexylphenyl)propenone.

HPLC-MS (Method A): m/z=347 (M+1); Rt=6.74 min

Mp=99-100° C.

MA: Calculated for C₂₅H₃₀O:

C, 86.66%; H, 8.73%; found:

C, 86.44%; H, 9.09%.

### 4-[5-(4-tert-Butylbenzoyl)-4-(4-cyclohexylphenyl)-4,5-dihydroisoxazol-3-yl]benzoic acid methyl ester and the regioisomer 4-[4-(4-tert-butylbenzoyl)-5-(4-cyclohexylphenyl)-4,5-dihydroisoxazol-3-yl]benzoic acid methyl ester

Dichloroisocyanuric acid sodium salt (1.7 g, 9 mmol) was dissolved in water (3 mL) and aluminiuoxide (3 g) was added. The mixture was evaporated to dryness and suspended in dichloromethane (10 mL). 4-(Hydroxyiminomethyl)benzoic acid methyl ester (0.54 g, 3.0 mmol) and 1-(4-tert-butylphenyl)-3-(4-cyclohexylphenyl)propenone (1.1 g, 3.0 mmol) was added and the mixture stirred at 5-8° C. for 3 hours. The mixture was filtered and filtrate concentrated in vacuo. The crude product was purified by flash chromatography on silica using ethyl acetate and heptane (gradient from 1:9 to 1:1) to give 0.12 g of 4-[5-(4-tert-butylbenzoyl)-4-(4-cyclohexylphenyl)-4,5-dihydroisoxazol-3-yl]benzoic acid methyl ester as the first isomer that was eluated and 0.12 g of 4-[4-(4-tert-butylbenzoyl)-5-(4-cyclohexylphenyl)-4,5-dihydroisoxazol-3-yl]benzoic acid methyl ester as the second isomer that was eluated.

### 4-[5-(4-tert-Butylbenzoyl)-4-(4-cyclohexylphenyl)isoxazol-3-yl]benzoic acid methyl ester

4-[5-(4-tert-Butylbenzoyl)-4-(4-cyclohexylphenyl)-4,5-dihydroisoxazol-3-yl]benzoic acid methyl ester (0.12 g, 0.23 mmol) was suspended in THF (2 mL) and DBU (0.123 mL, 0.82 mmol), and idodine (70 mg, 0.276 mmol) was added. After 2.5 hours, the reaction mixture was concentrated in vacuo and the residue was dissolved in dichloromethane (15 mL). The mixture was treated with sodium sulphite (6 mL of a saturated aqueous solution) and the aqueous phase was isolated and extracted with dichloromethane (10 mL). The organic phase was washed with 1 N hydrochloric acid, (6 mL), brine (6 mL), dried and concentrated in vacuo to give 0.13 g of crude product that was purified on silica using ethyl acetate and heptane (2:8) to give pure 4-[5-(4-tert-butylbenzoyl)-4-(4-cyclohexylphenyl)isoxazol-3-yl]benzoic acid methyl ester.

The structure of this compound was confirmed by NMR (NOE-experiment).

### 4-[5-(4-tert-Butylbenzoyl)-4-(4-cyclohexylphenyl)isoxazol-3-yl]benzoic acid

4-[5-(4-tert-Butylbenzoyl)-4-(4-cyclohexylphenyl)isoxazol-3-yl]benzoic acid methyl ester (0.029 g, 0.056 mmol) was dissolved in ethanol (1.2 mL) whereto aqueous sodium hydroxide (4N, 0.084 mL) was added. After 2 hours, the reaction mixture was concentrated in vacuo and the residue was dissolved in water (10 mL). The aqueous solution was acidified with 4N hydrochloric acid (0.1 mL) and extracted with ethyl acetate (2×10 mL). The organic phase was dried and concentrated in vacuo to give 0.28 g of crude product that was used in the next step.

HPLC-MS (Method A): m/z=508 (M+1); Rt=6.33 min

### 3-{4-[5-(4-tert-Butylbenzoyl)-4-(4-cyclohexylphenyl)isoxazol-3-yl]benzoylamino}propionic acid methyl ester

4-[5-(4-tert-Butylbenzoyl)-4-(4-cyclohexylphenyl)isoxazol-3-yl]benzoic acid (0.04 g, 0.079 mmol) was dissolved in DMF (2 mL) and HOAt (0.013 g, 0.095 mmol) and EDAC (0.018 g, 0.095 mmol) were added. After 30 minutes the hydrochloride of 3-aminopropionic acid methyl ester (0.017 g, 0.119 mmol) was added followed by addition of DIPEA (0.02 mL, 0.119 mmol). After 16 hours at room temperature the reaction mixture was diluted with ethyl acetate (10 mL) and washed with water (5 mL). The organic phase was washed with 0.2N hydrochloric acid (3×5 mL) and water (5 mL), dried, and concentrated in vacuo to give 0.05 g of crude 3-{4-[5-(4-tert-butylbenzoyl)-4-(4-cyclohexylphenyl)isoxazol-3-yl]benzoylamino}propionic acid methyl ester.

3-{4-[5-(4-tert-Butylbenzoyl)-4-(4-cyclohexylphenyl)isoxazol-3-yl]benzoylamino}propionic acid methyl ester (0.05 g) was dissolved in ethanol (2 mL) and aqueous sodium hydroxide (4N, 0.13 mL) was added. After 1.5 hours the reaction mixture was concentrated in vacuo and the residue was suspended in water (10 mL). The aqueous mixture was acidified with 4N hydrochloric acid (0.14 mL) and extracted with ethyl acetate (2×15 mL). The organic phase was washed with water (10 mL) and brine (10 mL), dried, and concentrated in vacuo. The crude product was purified on reverse phase HPLC (Waters Deltprep 4000 (gradient 30--->95% acetonitrile 40 min)) to give the title compound.

¹H-NMR (DMSO-d₆) selected peaks: δ=8.6 (t, 1H); 7.85 (d, 2H); 7.75 (d, 2H); 7.60 (d, 2H); 7.55 (d, 2H); 7.40 (d, 2H), 7.30 (d, 2H).

### Example 13

### 3-{4-[4-(4-tert-Butylbenzoyl)-5-(4-cyclohexylphenyl)isoxazol-3-yl]benzoylamino}propionic acid

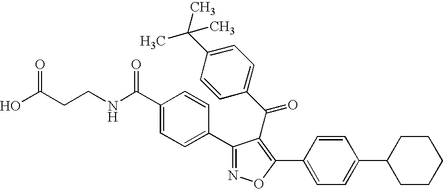

This compound was prepared analogously to the compound described in example 12, using the other isolated isomer.

### 4-[4-(4-tert-Butylbenzoyl)-5-(4-cyclohexylphenyl)isoxazol-3-yl]benzoic acid methyl ester

4-[4-(4-tert-Butylbenzoyl)-5-(4-cyclohexylphenyl)-4,5-dihydroisoxazol-3-yl]benzoic acid methyl ester (0.11 g, 0.21 mmol) was suspended in THF (2 mL) and DBU (0.113 mL, 0.76 mmol), and idodine (64 mg, 0.252 mmol) was added. After 3.5 hours the reaction mixture was concentrated in vacuo and the residue was dissolved in dichloromethane (10 mL). The mixture was treated with sodium sulphite (6 mL of a saturated aqueous solution) and the aqueous phase was extracted with dichloromethane (10 mL). The organic phase was washed with 1 N hydrochloric acid, (6 mL), brine (6 mL), dried and concentrated in vacuo to give 0.1 g of crude product that was purified by silica ge chromatography using ethyl acetate and heptane (2:8) to give pure 4-[4-(4-tert-butylbenzoyl)-5-(4-cyclohexylphenyl)isoxazol-3-yl]benzoic acid methyl ester.

The structure of this compound was confirmed by NMR (NOE-experiment).

### 4-[4-(4-tert-Butylbenzoyl)-5-(4-cyclohexylphenyl)isoxazol-3-yl]benzoic acid

4-[4-(4-tert-Butylbenzoyl)-5-(4-cyclohexylphenyl)isoxazol-3-yl]benzoic acid methyl ester (0.048 g, 0.092 mmol) was partly dissolved in ethanol (2 mL) whereto aqueous sodium hydroxide (4N, 0.14 mL) was added. After 2.5 hours the reaction mixture was concentrated in vacuo and the residue dissolved in water (10 mL). The aqueous solution was acidified with 4N hydrochloric acid (0.1 mL) and extracted with ethyl acetate (2×10 mL). The organic phase was dried and concentrated in vacuo to give 0.40 g of crude product that was used in the next step.

HPLC-MS (Method A): m/z=508 (M+1); Rt=6.39 min

### 3-{4-[4-(4-tert-Butylbenzoyl)-5-(4-cyclohexylphenyl)isoxazol-3-yl]benzoylamino}propionic acid methyl ester

4-[4-(4-tert-Butylbenzoyl)-5-(4-cyclohexylphenyl)isoxazol-3-yl]benzoic acid (0.03 g, 0.059 mmol) was dissolved in DMF (1.5 mL) and HOAt (0.01 g, 0.071 mmol) and EDAC (0.014 g, 0.071 mmol) were added. After 30 min the hydrochloride of 3-aminopropionic acid methyl ester (0.013 g, 0.09 mmol) was added followed by addition of DIPEA (0.015 mL, 0.09 mmol). After 16 hours at room temperature the reaction mixture was diluted with ethyl acetate (10 mL) and washed with water (5 mL). The organic phase was washed with 0.2N hydrochloric acid (3×5 mL) and water (5 mL), dried, and concentrated in vacuo to give 0.03 g of crude 3-{4-[4-(4-tert-butylbenzoyl)-5-(4-cyclohexylphenyl)isoxazol-3-yl]benzoylamino}propionic acid methyl ester.

HPLC-MS (Method A): m/z=593 (M+1); Rt=6.25 min

3-{4-[4-(4-tert-Butylbenzoyl)-5-(4-cyclohexylphenyl)isoxazol-3-yl]benzoylamino}propionic acid methyl ester (0.03 g) was dissolved in ethanol (1.5 mL) and aqueous sodium hydroxide (4N, 0.09 mL) was added. After 1 hour the reaction mixture was concentrated in vacuo and the residue was suspended in water (10 mL). The aqueous mixture was acidified with 4N hydrochloric acid (0.14 mL) and extracted with ethyl acetate (2×15 mL). The organic phase was washed with water (10 mL) and brine (10 mL), dried, and concentrated in vacuo to give the title compound.

HPLC-MS (Method A): m/z=579 (M+1); Rt=7.24 min

¹H-NMR (CDCl₃) selected peaks: δ=7.75-7.68 (m, 6H); 7.60 (d, 2H); 7.35 (d, 2H); 7.20 (d, 2H); 6.78 (br, 1H), 3.72 (m, 2H), 2.71 (m, 2H), 2.49 (m, 1H),

Pharmacological Methods

In the following section binding assays as well as functional assays useful for evaluating the efficiency of the compounds of the invention are described.

Binding of compounds to the glucagon receptor may be determined in a competition binding assay using the cloned human glucagon receptor.

Antagonism may be determined as the ability of the compounds to inhibit the amount of cAMP formed in the presence of 5 nM glucagon.

Glucagon Binding Assay (I)

Receptor binding are assayed using cloned human receptor (Lok et al., Gene 140, 203-209 (1994)). The receptor inserted in the pLJ6′ expression vector using EcoRI/SSt1 restriction sites (Lok et al.) is expressed in a baby hamster kidney cell line (A3 BHK 570-25). Clones are selected in the presence of 0.5 mg/mL G-418 and are shown to be stable for more than 40 passages. The K_{d} is shown to be 0.1 nM.

Plasma membranes are prepared by growing cells to confluence, detaching them from the surface and resuspending the cells in cold buffer (10 mM tris/HCl, pH 7.4 containing 30 mM NaCl, 1 mM dithiothreitol, 5 mg/l leupeptin (Sigma), 5 mg/l pepstatin (Sigma), 100 mg/l bacitracin (Sigma) and 15 mg/l recombinant aprotinin (Novo Nordisk A/S)), homogenization by two 10-s bursts using a Polytron PT 10-35 homogenizer (Kinematica), and centrifugation upon a layer of 41 w/v % sucrose at 95.000×g for 75 min. The white band located between the two layers is diluted in buffer and centrifuged at 40.000×g for 45 min. The precipitate containing the plasma membranes is suspended in buffer and stored at −80° C. until use.

Glucagon is iodinated according to the chloramine T method (Hunter and Greenwood, Nature 194, 495 (1962)) and purified using anion exchange chromatography (Jorgensen et al., Hormone and Metab. Res. 4, 223-224 (1972). The specific activity is 460 μCi/μg on the day of iodination. Tracer is stored at −18° C. in aliquots and used immediately after thawing.

Binding assays are carried out in triplicate in filter microtiter plates (MADV N65, Millipore). The buffer is 50 mM HEPES, 5 mM EGTA, 5 mM MgCl₂, 0.005% tween 20, pH 7.4. Glucagon is dissolved in 0.05 M HCl, added an equal amount (w/w) of human serum albumin and freeze-dried. On the day of use, it is dissolved in water and diluted in buffer to the desired concentrations.

Test compounds are dissolved and diluted in DMSO. 140 μl buffer, 25 μl glucagon or buffer, and 10 μl DMSO or test compound are added to each well. Tracer (50.000 cpm) is diluted in buffer and 25 μl is added to each well. 1-4 μg freshly thawed plasma membrane protein diluted in buffer is then added in aliquots of 25 μl to each well. Plates are incubated at 30° C. for 2 hours. Non-specific binding is determined with 10-6 M of glucagon. Bound tracer and unbound tracer are then separated by vacuum filtration (Millipore vacuum manifold). The plates are washed with 2×100 μl buffer/well. The plates are air dried for a couple of hours, whereupon the filters are separated from the plates using a Millipore Puncher. The filters are counted in a gamma counter.

Functional Assay (I)

The functional assay is carried out in 96 well microtiter plates (tissue culture plates, Nunc). The resulting buffer concentrations in the assay are 50 mM tris/HCl, 1 mM EGTA, 1.5 mM MgSO₄, 1.7 mM ATP, 20 μM GTP, 2 mM IBMX, 0.02% tween-20 and 0.1% human serum albumin. pH was 7.4. Glucagon and proposed antagonist are added in aliquots of 35 μl diluted in 50 mM tris/HCl, 1 mM EGTA, 1.85 mM MgSO₄, 0.0222% tween-20 and 0.111% human serum albumin, pH 7.4.20 μl of 50 mM tris/HCl, 1 mM EGTA, 1.5 mM MgSO₄, 11.8 mM ATP, 0.14 mM GTP, 14 mM IBMX and 0.1% human serum albumin, pH 7.4 was added. GTP was dissolved immediately before the assay.

50 μl containing 5 μg of plasma membrane protein was added in a tris/HCl, EGTA, MgSO₄, human serum albumin buffer (the actual concentrations are dependent upon the concentration of protein in the stored plasma membranes).

The total assay volume is 140 μl. The plates are incubated for 2 hours at 37° C. with continuous shaking. Reaction is terminated by addition of 25 μl 0.5 N HCl. cAMP is measured by the use of a scintillation proximity kit (Amersham).

Glucagon Binding Assay (II)

BHK (baby hamster kidney cell line) cells are transfected with the human glucagon receptor and a membrane preparation of the cells is prepared. Wheat Germ Agglutinin derivatized SPA beads containing a scintillant (WGA beads) (Amersham) bound the membranes. ¹²⁵I-glucagon bound to human glucagon receptor in the membranes and excited the scintillant in the WGA beads to light emission. Glucagon or samples binding to the receptor competed with ¹²⁵I-glucagon.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000×g) the supernatant is transferred to cold centrifuge tubes and centrifuged for 45 min at 40.000×g. The pellet is resuspended in homogenisation buffer, homogenised 2×10 sec (Polytron) and additional homogenisation buffer is added. The suspension is centrifuged for 45 min at 40.000×g and the pellet is resuspended in resuspension buffer (25 mM HEPES, pH=7.4, 2.5 mM CaCl₂, 1.0 mM MgCl₂) and homogenised 2×10 sec. (Polytron).

The protein concentration is normally around 1.75 mg/mL. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The glucagon binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl glucagon or test compound (in DMSO) are added to each well. 50 μl tracer (125I-porcine glucagon, 50.000 cpm) and 50 μl membranes (7.5 μg) containing the human glucagon receptor are then added to the wells. Finally 50 μl WGA beads containing 1 mg beads are transferred to the well. The opti plates are incubated for 4 hours on a shaker and then settled for 8-48 hours. The opti plates are counted in a Topcounter. Non-specific binding is determined with 500 nM of glucagon.

Most of the compounds according to the examples showed IC₅₀ values below 1000 nM when tested in the glucagon binding assay (II).

GIP Binding Assay

BHK (baby hamster kidney cell line) cells are transfected with the human GIP receptor and a membrane preparation of the cells is prepared. Wheat Germ Agglutinin derivatized SPA beads containing a scintillant (WGA beads) (Amersham) bound the membranes. ¹²⁵I-GIP bound to human GIP receptor in the membranes and excited the scintillant in the WGA beads to light emission. GIP or samples binding to the receptor competed with ¹²⁵I-GIP.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000×g) the supernatant is transferred to cold centrifuge tubes and centrifuged for 45 min at 40.000×g. The pellet is resuspended in homogenisation buffer, homogenised 2×10 sec (Polytron) and additional homogenisation buffer is added. The suspension is centrifuged for 45 min at 40.000×g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/mL. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The GIP binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl GIP or test compound (in DMSO) are added to each well. 50 μl tracer (¹²⁵I-porcine GIP, 50.000 cpm) and 50 μl membranes (20 μg) containing the human GIP receptor are then added to the wells. Finally 50 μl WGA beads containing 1 mg beads are transferred to the well. The opti plates are incubated for 3.5 hours on a shaker and then settled for 8-48 hours. The opti plates are counted in a Topcounter. Non-specific binding is determined with 500 nM of GIP.

Generally, the compounds show a higher affinity for the glucagon receptor compared to the GIP receptor.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

1. A compound of the general formula (I): 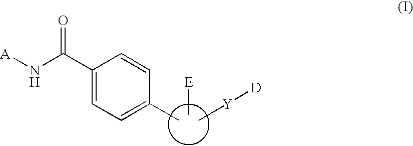
wherein
A is 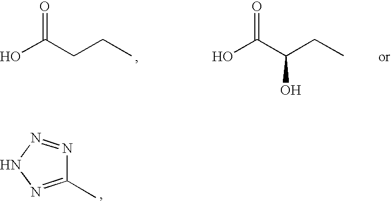
Y is a valence bond, >C═O, ═CR¹—, —(CR¹R²)ₘ—, —NR¹—, ═N—,
wherein R¹ and R² are independently selected from H and lower alkyl;
m is selected from 1, 2, 3, 4, 5 or 6;
E is
C₁₋₁₀-alkyl or C₂₋₁₀-alkenyl,
C₃₋₁₀-cycloalkyl, C₃₋₁₀-cycloalkenyl, C₇₋₁₀-bicycloalkyl, C₃₋₁₀-cycloalkyl-C₁₋₆-alkyl, C₃₋₁₀-cycloalkenyl-C₁₋₆-alkyl or C₇₋₁₀-bicycloalkyl-C₁₋₆-alkyl,
wherein the rings may optionally be substituted with one or more substituents selected from halogen, C₁₋₆-alkyl, C₂₋₆-alkenyl, C₁₋₆-alkoxy, C₁₋₆-thioalkyl, —CF₃, —OCF₃, —SCF₃, —OCHF₂ and —SCHF₂,
aryl, aryloxy, arylthio, heteroaryl, aryl-C₁₋₆-alkyl, aryloxy-C₁₋₆-alkyl, arylthio-C₁₋₆-alkyl, heteroaryl-C₁₋₆-alkyl, diaryl-C₁₋₆-alkyl or (C₁₋₆-alkyl)(aryl)-C₁₋₇-alkyl, wherein the non-aromatic and aromatic rings may optionally be substituted with one or more substituents selected from halogen, C₁₋₆-alkyl, C₂₋₆-alkenyl, C₁₋₆-alkoxy, C₁₋₆-thioalkyl, —CF₃, —OCF₃, —SCF₃, —OCHF₂, —SCHF₂, C₃₋₁₀-cycloalkyl and C₃₋₁₀-cyclo-alkenyl, or with two substituents on adjacent positions which are combined to form a bridge C₁₋₆-alkylene, C₂₋₆-alkenylene or —O—C₁₋₆-alkylene-O—, 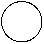
represents a phenyl, C₃₋₈-cycloalkyl, or a 5-, 6- or 7-membered heterocycle, and the rings are optionally substituted with one or two substituents selected from C₁₋₆-alkyl or hydroxy, which may give a keto-group depending on tautomerism,
D is aryl or heteroaryl,
which may optionally be substituted with one or more substituents selected from
halogen, —CF₃, —OCF₃, —SCF₃, —CN, —NO₂, C₁₋₁₀-alkyl, C₂₋₆-alkenyl, C₁₋₆-alkoxy, C₁₋₆-alkylthio, amino, C₁₋₆-alkylamino, di-C₁₋₆-alkylamino, —SO₂CF₃ and —SO₂—C₁₋₆-alkyl,
C₃₋₈-cycloalkyl, C₃₋₈-cycloalkenyl, aryl and aryl-C₁₋₆-alkoxy, wherein the non-aromatic and aromatic rings optionally may be substituted with one to three substituents selected from halogen, —CF₃, —OCF₃, —SCF₃, —CN, —NO₂, C₁₋₁₀-alkyl, C₂₋₆-alkenyl, C₁₋₆-alkoxy and C₁₋₆-alkylthio, or with two substituents on adjacent positions which are combined to form a bridge —O—(CH₂)₈—O—(CH₂)ₚ— or —O—(CF₂)ₙ—O—(CF₂)ₚ—, wherein s is an integer of from 1 to 6, and p is 0 or 1,
or with two substituents on adjacent positions which are combined to form a bridge —O—(CH₂)ₛ—O—(CH₂)ₚ— or —O—(CF₂)ₛ—O—(CF₂)ₚ—, wherein s is an integer of from 1 to 6, and p is 0 or 1,
as well as any diastereomer or enantiomer or tautomeric form thereof including mixtures of these or a pharmaceutically acceptable salt thereof.

2. A compound according to claim 1, wherein
E is
C₁₋₁₀-alkyl or C₂₋₁₀-alkenyl,
C₃₋₁₀-cycloalkyl or C₃₋₁₀-cycloalkenyl, which may optionally be substituted with one or two substituents selected from halogen, C₁₋₆-alkyl, C₁₋₆-alkoxy, C₁₋₆-thioalkyl, —CF₃, —OCF₃, —SCF₃, —OCHF₂ and —SCHF₂, 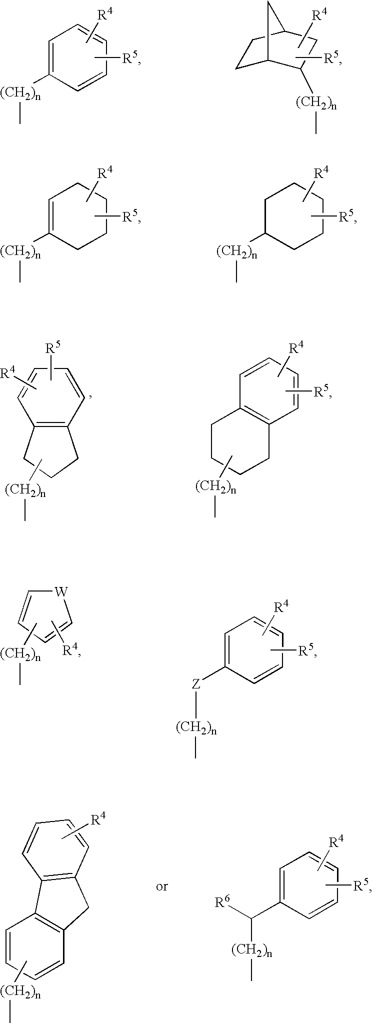
R⁴ and R⁵ independently are hydrogen, halogen, C₁₋₆-alkyl, C₂₋₆-alkenyl, C₁₋₆-alkoxy, C₁₋₆-thioalkyl, —CF₃, —OCF₃, —SCF₃, —OCHF₂, —SCHF₂, C₃₋₁₀-cycloalkyl or C₃₋₁₀-cyclo-alkenyl, or R⁴ and R⁵ on adjacent positions may be combined to form a bridge —O—C₁₋₆-alkylene-O—, C₁₋₈-alkylene or C₃₋₈-alkenylene,
R⁶ is C₁₋₆-alkyl or aryl, wherein aryl may optionally be substituted with one or two substituents selected from halogen, C₁₋₆-alkyl, C₂₋₆-alkenyl, C₁₋₆-alkoxy, C₁₋₆-thioalkyl, —CF₃, —OCF₃, —SCF₃, —OCHF₂ and —SCHF₂,
n is an integer of from 0 to 6,
Z is —O— or —S—,
W is —O—, —S—, or —NR⁷—,
R⁷ is hydrogen or C₁₋₆-alkyl,
D is 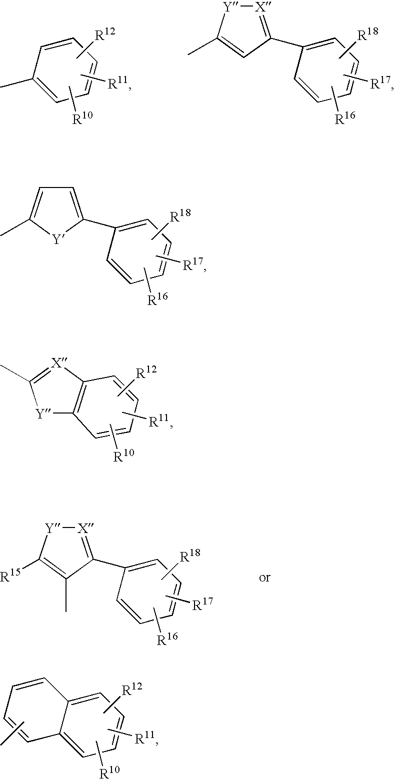
R¹⁰, R¹¹ and R¹² independently are
hydrogen, halogen, —CF₃, —OCF₃, —SCF₃, —CN, —NO₂, C₁₋₁₀-alkyl, C₂₋₆-alkenyl, C₁₋₆-alkoxy, C₁₋₆-alkylthio, amino, C₁₋₆-alkylamino, di-C₁₋₆-alkylamino, —SO₂CF₃ or —SO₂—C₁₋₆-alkyl,
C₃₋₈-cycloalkyl, C₃₋₈-cycloalkenyl, aryl or aryl-C₁₋₆-alkoxy,
wherein the non-aromatic and aromatic rings optionally may be substituted with one to three substituents selected from halogen, —CF₃, —OCF₃, —SCF₃, —CN, —NO₂, C₁₋₁₀-alkyl, C₂₋₆-alkenyl, C₁₋₆-alkoxy and C₁₋₆-alkylthio, or with two substituents on adjacent positions which are combined to form a bridge —O—(CH₂)₈—O—(CH₂)ₚ— or —O—(CF₂)ₙ—O—(CF₂)ₚ—, wherein s is an integer of from 1 to 6, and p is 0 or 1,
or two of R¹⁰, R¹¹ and R¹² on adjacent positions are combined to form a bridge —O—(CH₂)ₛ—O—(CH₂)ₚ— or —O—(CF₂)ₛ—O—(CF₂)ₚ—, wherein s is an integer of from 1 to 6, and p is 0 or 1,
X″ is —N═ or —CR¹³═,
Y″ is —S—, —O— or —NR¹⁴—,
R¹³ and R¹⁵ independently are hydrogen, C₁₋₆-alkyl or aryl, wherein aryl is optionally substituted with one or two substituents selected from halogen, C₁₋₆-alkyl, C₁₋₆-alkoxy, C₁₋₆-thioalkyl, —CF₃, —OCF₃, —SCF₃, —OCHF₂ and —SCHF₂,
R¹⁴ is hydrogen or C₁₋₆-alkyl,
R¹⁶, R¹⁷ and R¹³ independently are hydrogen, halogen, —CF₃, —OCF₃, —SCF₃, —CN, —NO₂, C₁₋₁₀-alkyl, C₂₋₆-alkenyl, C₁₋₆-alkoxy and C₁₋₆-alkylthio, or with two substituents on adjacent positions which are combined to form a bridge —O—(CH₂)_{q}—O—(CH₂)ᵣ— or —O(CF₂)_{q}—O—(CF₂)ᵣ—, wherein q is an integer of from 1 to 6, and r is 0 or 1,
as well as any diastereomer or enantiomer or tautomeric form thereof including mixtures of these or a pharmaceutically acceptable salt thereof.

3. A compound according to claim 1, wherein A is —(CH₂)₂—COOH.

4. A compound according to claim 1, wherein Y is a valence bond, >C═O, ═CR¹—, —(CR¹R²)ₘ—, —, ═N—,
R¹ and R² are independently selected from H and lower alkyl; and
m is selected from 1, 2, 3.

5. A compound according to claim 1, wherein 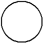
s cyclopropyl, phenyl, furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl or thiadiazinyl, including the fully or partially saturated analogues and C₁₋₆-alkyl- and hydroxy substituted derivatives of any of the above.

6. A compound according to claim 5, wherein 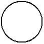
is cyclopropyl, oxazolyl, isoxazolyl, pyridazinyl, pyrrolyl, thiazolyl, 1,2,4-triazolyl, pyrazolyl, imidazolyl, all of which includes the fully or partially saturated analogues and C₁₋₆-alkyl and hydroxy substituted derivatives of any of the above.

7. A compound according to claim 6 wherein 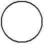
is

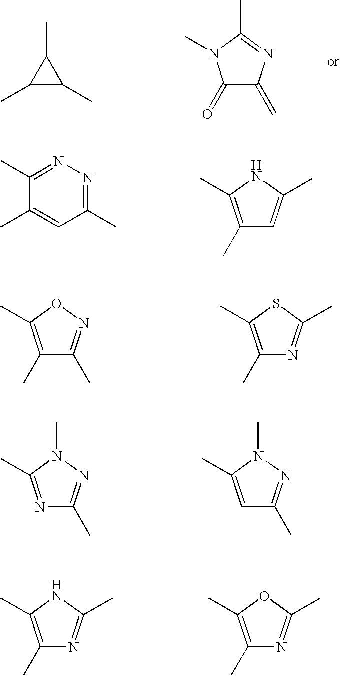

8. A compound according to claim 7, wherein 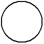
is

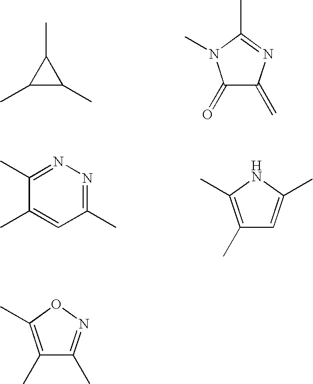

9. A compound according to claim 1, wherein E is
C₁₋₁₀-alkyl,
C₃₋₁₀-cycloalkyl, which may optionally be substituted as defined in claim 1, 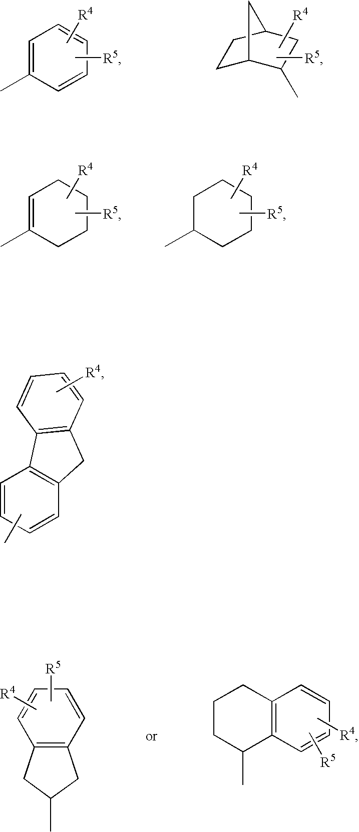
and wherein R⁴ and R⁵ are as defined in claim 1.

10. A compound according to claim 9, wherein E is
C₁₋₁₀-alkyl,
C₃₋₁₀-cycloalkyl, 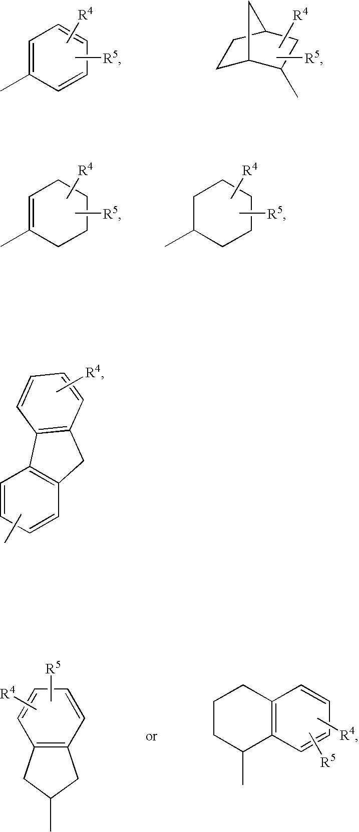
wherein R⁴ and R⁵ independently are hydrogen, halogen, C₁₋₆-alkyl, C₁₋₆-alkoxy, —OCF₃, —CF₃, —SCF₃, C₃₋₁₀-cycloalkyl or C₃₋₁₀-cycloalkenyl, or R⁴ and R⁵ on adjacent positions may be combined to form a bridge C₁₋₆-alkylene or C₂₋₆-alkenylene.

11. A compound according to claim 10, wherein E is 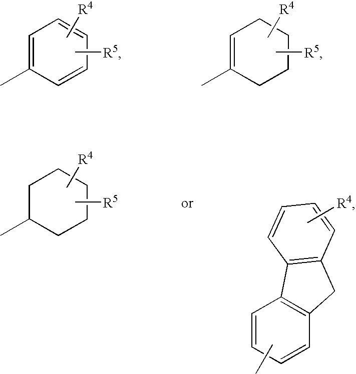
wherein R⁴ and R⁵ independently are hydrogen, halogen, C₁₋₆-alkyl, C₁₋₆-alkoxy, —OCF₃, —CF₃, —SCF₃, C₃₋₁₀-cycloalkyl or C₃₋₁₀-cycloalkenyl, or R⁴ and R⁵ on adjacent positions may be combined to form a bridge C₁₋₆-alkylene or C₂₋₆-alkenylene.

12. A compound according to claim 11 wherein E is 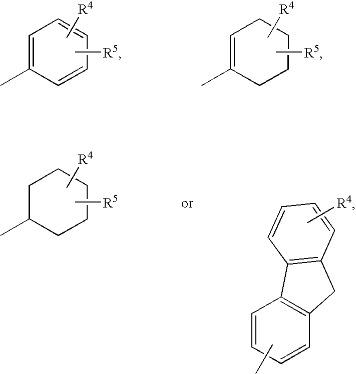
wherein R⁴ and R⁵ independently are hydrogen, halogen, C₁₋₆-alkyl, C₁₋₆-alkoxy, —OCF₃, —CF₃, —SCF₃, cyclohexyl or cyclohex-1-enyl, or R⁴ and R⁵ on adjacent positions may be combined to form a bridge C₁₋₆-alkylene.

13. A compound according to claim 12 wherein E is 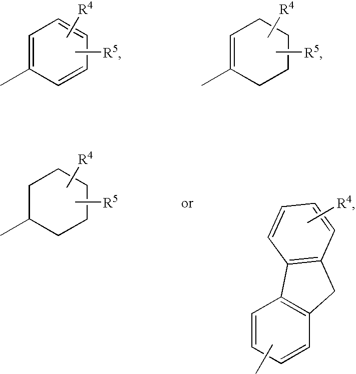
wherein R⁴ is hydrogen and R⁵ is C₁₋₆-alkyl, cyclohexyl, halogen, —CF₃ or cyclohex-1-enyl,
or R⁴ and R⁵ on adjacent positions may be combined to form a bridge C₁₋₆-alkylene.

14. A compound according to claim 13 wherein E represents 4-trifluoromethoxyphenyl, 4-cyclohexylphenyl or biphenyl-4-yl;

15. A compound according to claim 1, wherein E is 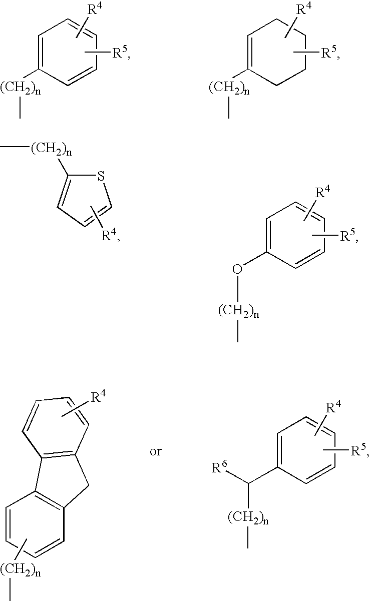
wherein n is 1, 2 or 3, and R⁴, R⁵ and R⁶ are as defined in claim 1.

16. A compound according to claim 15, wherein R⁴ and R⁵ independently are hydrogen, halogen, —OCF₃, —CF₃, C₁₋₆-alkoxy or C₂₋₆-alkenyl, or
R⁴ and R⁵ on adjacent atoms together form the bridge —O—CH₂—O—.

17. A compound according to claim 1, wherein D is 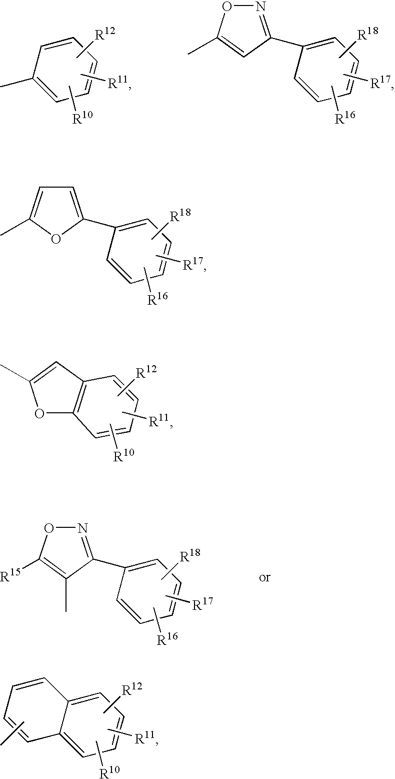
wherein R¹⁰, R¹¹, R¹², R¹⁵, R¹⁶, R¹⁷ and R¹⁸ are as defined in claim 1.

18. A compound according to claim 1, wherein D is 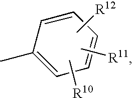
wherein R¹⁰, R¹¹ and R¹² are as defined in claim 1.

19. A compound according to claim 17, wherein R¹⁰, R¹¹ and R¹² independently are hydrogen, halogen, —OCF₃, —CF₃, —NO₂, di-C₁₋₆-alkylamino, C₁₋₁₀-alkyl, C₁₋₆-alkoxy or —CN,
phenyl or phenyl-C₁₋₆-alkoxy, which may optionally be substituted with one or two substituents as defined in claim 1,
or two of R¹⁰, R¹¹ and R¹² in adjacent positions form a bridge —O—CH₂—O—, —O—CH₂—CH₂—O—, —O—CH₂—CH₂—CH₂—O—, —O—CF₂—O—, —O—CF₂—O—CF₂— or —O—CF₂—CF₂—O—.

20. A compound according to claim 19, wherein R¹⁰, R¹¹ and R¹² independently are hydrogen, halogen, —OCF₃, —CF₃, —NO₂, di-C₁₋₆-alkylamino, C₁₋₁₀-alkyl, C₁₋₆-alkoxy or —CN,
phenyl or phenyl-C₁₋₆-alkoxy,
or two of R¹⁰, R¹¹ and R¹² in adjacent positions form a bridge —O—CH₂—O—, —O—CH₂—CH₂—O— or —O—CH₂—CH₂—CH₂—O—.

21. A compound according to claim 20, wherein two of R¹⁰ and R¹¹ are hydrogen, and and R¹² is halogen, —OCF₃, —CF₃, —NO₂, di-C₁₋₆-alkylamino, C₁₋₁₀-alkyl, C₁₋₆-alkoxy or —CN.

22. A compound according to claim 1, wherein Y-D represents 4-trifluoromethoxybenzylidene, 3,5-dichlorobenzylidene, 4-trifluoromethoxyphenyl, 3,5-Bis-trifluoromethylphenyl, 4-trifluoromethylsulfanyl-phenyl, 4-trifluoromethoxybenzoyl or 4-tert-Butylbenzoyl;

23. A compound of the general formula (I₁):

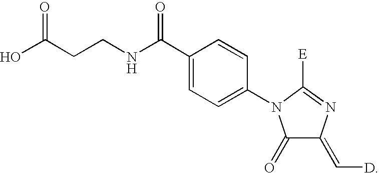

24. A compound of the general formula (I₃): 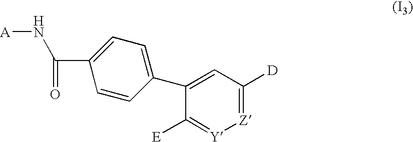
wherein R^{x} represents H or OH, and —Y′=Z′- (or ═Y′-Z′=) is —N═N- (or ═N—N═), —O—, —S—, —NR′, wherein R′ is hydrogen, lower alkyl, lower alkoxy, hydroxy, amino, lower alkylaryl, or aryl and E and D are as defined in any one of the preceding claims, as well as any diastereomer or enantiomer or tautomeric form thereof including mixtures of these or a pharmaceutically acceptable salt thereof.

25. A compound according to claim 24, of the general formula: 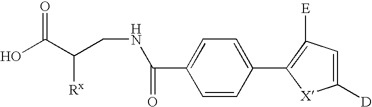
wherein X′ is —O—, —S—, —NR′—, wherein R′ is hydrogen, lower alkyl, lower alkoxy, hydroxy, amino, lower alkylaryl, or aryl and E and D are as defined in any one of the preceding claims, as well as any diastereomer or enantiomer or tautomeric form thereof including mixtures of these or a pharmaceutically acceptable salt thereof.

26. A compound of the general formula (I₄): 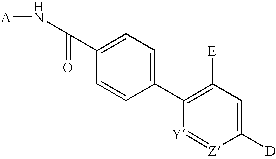
wherein R^{x} represents H or OH, and -′Y=Z′- (or ═Y′-Z′=) is —N═N- (or ═N—N═), —O—, —S—, —NR′, wherein R′ is hydrogen, lower alkyl, lower alkoxy, hydroxy, amino, lower alkylaryl, or aryl and E and D are as defined in any one of the preceding claims, as well as any diastereomer or enantiomer or tautomeric form thereof including mixtures of these or a pharmaceutically acceptable salt thereof.

27. A compound according to claim 1, which has an IC₅₀ value of no greater than 5 μM as determined by the Glucagon Binding Assay (I) or Glucagon Binding Assay (II) disclosed herein.

28. A compound according to claim 27, which has an IC₅₀ value of less than 1 μM, preferably of less than 500 nM and even more preferred of less than 100 nM as determined by the Glucagon Binding Assay (I) or Glucagon Binding Assay (II) disclosed herein.

29. A pharmaceutical composition comprising, as an active ingredient, a compound according to claim 1.

30. A method for the treatment of disorders or diseases, wherein a glucagon antagonistic action is beneficial, the method comprising administering to a subject in need thereof an effective amount of a compound according to claim 1 or a pharmaceutical composition according to claim 29.
